SEQUENCE LISTING (2 sequences)

SEQ ID NO: 1 - protein, 18 aa, synthetic construct
  REGION 1..18: note = STAT3 Inhibitor Peptide
  MOD_RES 2: note = amino acid at position 2 is phosphorylated Tyrosine (Tyr(PO3H2))
PXLKTKAAVLLPVLLAAP

SEQ ID NO: 2 - protein, 28 aa, synthetic construct
  REGION 1..28: note = STAT3 Inhibitor XII SPI
FISKERERAILSTKPPGTFLLRFSESSK

(19) United States
(12) Patent Application Publication
(10) Pub. No.: US 2026/0232752 A1
(43) Pub. Date: Aug. 13, 2026

(54) COMBINATION THERAPY METHODS, COMPOSITIONS AND KITS

(71) Applicants: Accure Therapeutics, S.L., Barcelona (ES); Fundació De Recerca Clínic Barcelona-Institut D’Investigacions Biomèdiques August PI I Sunyer, Barcelona (ES)

(72) Inventor: Pablo Villoslada Diaz, Los Altos, CA (US)

(73) Assignees: Accure Therapeutics, S.L., Barcelona (ES); Fundació De Recerca Clínic Barcelona-Institut D’Investigacions Biomèdiques August PI I Sunyer, Barcelona (ES)

(21) Appl. No.: 19/652,855

(22) Filed: Apr. 20, 2026

Related U.S. Application Data
(63) Continuation of application No. 17/622,660, filed on Jul. 2, 2020, now abandoned.

(30) Foreign Application Priority Data
Jul. 3, 2019 (EP) 19382566.8

(51) Int. Cl. A61K 38/06; A61K 31/137; A61K 31/167; A61K 31/18; A61K 31/225; A61K 31/381; A61K 31/397; A61K 31/4015; A61K 31/409; A61K 31/4245; A61K 31/426; A61K 31/44; A61K 31/47; A61K 31/4709; A61K 38/53; A61P 25/28

(52) U.S. Cl. CPC A61K 38/06; A61K 31/137; A61K 31/167; A61K 31/18; A61K 31/225; A61K 31/381; A61K 31/397; A61K 31/4015; A61K 31/409; A61K 31/4245; A61K 31/426; A61K 31/44; A61K 31/47; A61K 31/4709; A61K 38/53; A61P 25/28

(57) ABSTRACT

The invention relates to a method of treating optic neuritis comprising administering to a human in need thereof:
(a) a first pharmaceutical composition comprising:

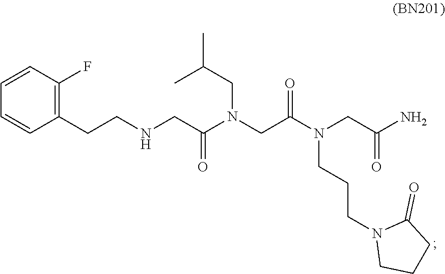 [SMILES: CC(C)CN(CC(=O)N(CCCN1CCCC1=O)CC(N)=O)C(=O)CNCCc1ccccc1F]

or a pharmaceutically acceptable salt thereof comprising at least one pharmaceutically acceptable excipient;
in a therapeutically effective combination with
(b) a second pharmaceutical composition of dimethyl fumarate or monomethyl fumarate, or a mixture thereof, or a pharmaceutically acceptable salt thereof, comprising at least one pharmaceutical excipient.

### CROSS-REFERENCE

This application is a continuation of U.S. patent application Ser. No. 17/622,660, filed Dec. 23, 2021, which is a 35 U.S.C. § 371 national-phase filing of International Application No. PCT/EP2020/068604, filed Jul. 2, 2020, which claims the benefit of European Patent Application 19382566.8, filed Jul. 3, 2019. The entirety of each of these applications is incorporated herein by reference for all purposes.

### TECHNICAL FIELD

The present invention relates to the field of the field of neurological diseases and to particular approaches for treating them using combination therapies.

### INCORPORATION BY REFERENCE

The Sequence Listing XML file named “23061-015WO1US2_ST26” created on Apr. 20, 2025, and having a size of 4,055 bytes, is hereby incorporated by reference.

### BACKGROUND ART

Inflammatory neurological diseases or conditions which can result in destruction or degeneration of axons or myelin may include, but are not limited to, various Central Nervous System (CNS) diseases such as multiple sclerosis (MS), Neuromyelitis optica (NMO), optic neuritis, Balo disease, Schilder's disease, transverse myelitis, acute hemorrhagic leukoencephalitis (i.e., Hurst's disease), and Marburg disease (i.e., acute MS).

MS is a degenerative autoimmune disease of the central nervous system (CNS) in which the immune system attacks and damages axons and the myelin protective sheath surrounding nerve fibers, resulting in significant disability. MS is characterized by demyelination, multifocal inflammation, reactive gliosis, and oligodendrocyte and axonal loss. There are three clinical disease courses that people affected with MS typically experience: relapsing-remitting MS (RRMS), secondary-progressive MS (SPMS), primary-progressive MS (PPMS). RRMS is the most common disease course, which affects approximately 85% of people with MS, and is characterized by clearly defined attacks (i.e., relapses) of worsening neurologic function. These relapses are followed by partial or complete recovery periods, during which symptoms improve partially or completely and there is no progression of disease. Most people with RRMS will eventually transition to SPMS, which means that after a period of time in which they experience relapses and remissions, the disease will begin to progress more steadily, with or without any relapses. PPMS affects about 10% of people with MS, and is characterized by steadily worsening neurologic function from the beginning in which there are no distinct relapses or remissions. The symptoms, severity, and course of MS will vary depending on the sites of the damaged myelin and the extent of demyelination.

NMO (also known as Devic's disease or Devic's syndrome) or NMO spectrum disorders (NMOSD) are autoimmune disorders of the CNS in which immune system cells and antibodies mistakenly attack and destroy astrocytes in the optic nerves, brain and the spinal cord, inducing secondary demyelination and axonal loss. Damage to the optic nerves causes optic neuritis which produces swelling and inflammation that causes pain and loss of vision. Damage to the spinal cord causes weakness or paralysis in the legs or arms, loss of sensation, and problems with bladder and bowel function. Because both diseases have similar symptoms and can cause attacks of optic neuritis and myelitis, NMO can be confused with MS, and until recently, was thought to be a severe variant of MS. However, recent studies suggest that NMO and MS are distinct diseases.

Optic neuritis is a demyelinating inflammation of the optic nerve that can be caused by many different conditions, but often occurs in association with MS and NMO. Inflammation can cause loss of vision or even blindness, often because of the swelling and destruction of the myelin sheath covering the optic nerve. Symptoms of optic neuritis include blurred vision, dimming of colors, pain when the eye is moved, blind spots and loss of contrast sensitivity.

Currently there is no cure for MS. The treatments that are available are directed to a subset of patients with relapsing forms of MS, which include RRMS, and those patients with SPMS who experience relapses. Most of the United States Food and Drug Administration (FDA) approved treatments are immunomodulatory drugs that decrease the frequency of relapses and delay the progression of MS. These treatments, however, are only partially effective and only target immune system activation without exerting neuroprotective or regenerative effects. Further, the current therapies are associated with significant side effects, such as adverse immune reactions or severe opportunistic infections.

At this moment, there is also no cure for NMO. Standard of care for NMO include intravenous high-dose corticosteroid treatment, plasmapheresis for treating relapses and rituximab, azathiorpine and micophenolate for relapse prevention, which can have serious side effects, including infection. Likelihood of recurrence of NMO is greater than 90 percent and attacks are generally severe; therefore, ongoing treatment to suppress the immune system is considered necessary. Thus, there is a need in the art for more effective methods of treating inflammatory neurological diseases or conditions which can result in the destruction or degeneration of axons or myelin such as MS, NMO, optic neuritis, Balo disease, Schilder's disease, transverse myelitis, acute hemorrhagic leukoencephalitis (i.e., Hurst's disease), and Marburg disease (acute MS).

### SUMMARY OF INVENTION

Inventors have found that combinations that comprise a compound of formula (I) (see below) and one or more drugs with immunomodulatory activity and/or anti-oxidant effects, and/or anti-inflammatory activity, provided meaningful therapeutic activity in inflammatory neurological diseases or conditions which can result in the destruction or degeneration of axons or myelin in a subject. Surprisingly, said combined use of the compounds enhanced or boosted the desired effect even when at least one of the compounds of formula (I) or the one or more additional drugs were administered at doses considered and classified as subtherapeutic amounts. The combination supposed improved therapeutic effects and also meaningful lowering of associated side-effects. More surprisingly was the fact that, these effects of meaningful therapeutic activity in inflammatory neurological diseases or conditions which can result in the destruction or degeneration of axons or myelin in a subject, were achieved even at suboptimal or subtherapeutic doses of each of the compounds when administered as single active agents. Thus, particular combinations of compounds of formula (I) with other particular compounds, exhibited a significantly greater improvement at subtherapeutic doses in these diseases than when administering each compound alone. Moreover, combinations of the compounds of formula (I) and the one or more additional drugs administered at doses lower than those expected to be active, were effective in a chronic phase or advanced phase or stage of the inflammatory neurological disease or condition.

This improved and unexpected effect when compounds were used in combination, has been shown in an animal model widely employed in inflammatory neurological diseases, the experimental autoimmune encephalomyelitis (EAE) in mice. According to the results, the inventors found that when used in combination, the compounds of formula (I) and the one or more drugs with immunomodulatory and/or anti-oxidant and/or anti-inflammatory activity, were able to improve the clinical score of EAE mice, in such a way that those disabling effects of the neurological diseases were lowered. This potentiated or improved effects are thought to be due to the activation of different biological pathways by each drug that converge in prevention of cascades leading to cell damage, such as oxidative stress, apoptosis, autophagy, synaptic pruning, energetic metabolism balance, etc.

Therefore, a first aspect of the invention relates to a combination comprising:
a) a compound of formula (I) or a pharmaceutically or veterinary acceptable salt thereof

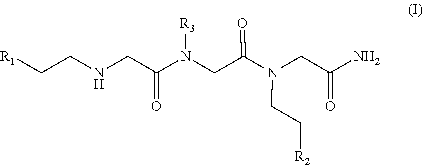

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl; and
b) one or more drugs selected from the group consisting of:
i) a compound of formula (IV), or a pharmaceutically or veterinary acceptable salt thereof

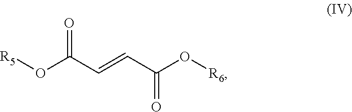

wherein R₅ is selected from hydrogen (H) and (C₁-C₆)alkyl, R₆ is selected from H, (C₁-C₆)alkyl and 2-(2,5-dioxopyrrolidin-1-yl)ethyl, and wherein if R₅ is H, R₆ is other than H,
ii) a sphingosine-1-phosphate receptor modulator (S1PR modulator), and
iii) a Signal transducer and activator of transcription 3 (STAT3) inhibitor.

The compounds of the invention may be formulated in different types of compositions/kits of parts. Thus, a second aspect of the invention relates to a single pharmaceutical or veterinary composition which comprises:
a) a compound of formula (I) or a pharmaceutically or veterinary acceptable salt thereof, and
b) one or more drugs selected from the group consisting of: i) a compound of formula (IV), or a pharmaceutically or veterinary acceptable salt thereof, ii) a S1PR modulator, and iii) a STAT3 inhibitor;
together with one or more pharmaceutically or veterinary acceptable excipients or carriers; wherein the compound of formula (I) and the drugs are as defined in the first aspect, and wherein the amount of a) and the amount of b) in combination are therapeutically effective.

A third aspect of the invention relates to a package or kit of parts comprising:
i) a first pharmaceutical or veterinary composition which comprises an amount of a compound of formula (I) as defined above, or a pharmaceutically or veterinary acceptable salt thereof, together with one or more pharmaceutically or veterinary acceptable excipients or carriers;
ii) a second pharmaceutical or veterinary composition which comprises an amount of one or more drugs selected from the group consisting of a compound of formula (IV), or a pharmaceutically or veterinary acceptable salt thereof, a S1PR modulator, and a STAT3 inhibitor, together with one or more pharmaceutically or veterinary acceptable excipients or carriers;
iii) instructions for the use in combination of i) and ii);
wherein the first and second compositions are separate compositions, and wherein the amount of the compound of formula (I) of i) and the amount of one or more drugs of ii) in combination are therapeutically effective.

Further, as mentioned above, the combination of the invention may be used in inflammatory neurological diseases or conditions which can result in the destruction or degeneration of axons or myelin.

Thus, a fourth aspect of the invention relates to the combination, the single pharmaceutical or veterinary composition, or the package or kit of parts as previously defined, for use in the treatment and/or prevention of inflammatory neurological diseases or conditions which can result in the destruction or degeneration of axons or myelin.

### BRIEF DESCRIPTION OF DRAWINGS

FIG. 1 (A), related with Example 1 shows clinical score (CS) per day of study EAE-C03, where effect of daily treatment with BN201 at different doses was compared to placebo. After 17 days of treatment, clinical score started to be significantly lower in treated group with BN201 (100 mg/kg) and BN201 (50 mg/kg) than in pathological control group (** p≤0.01; *p≤0.05). FIG. 1 (B), related with Example 1 shows clinical score (CS) per day study EAE-C05, where effect of daily treatment with BN201 at different doses and 2 active comparators (dimethyl fumarate DMF and fingolimod FTY720) were compared to placebo. After 5 days of treatment, clinical score started to be significantly lower in treated group with BN201 (100 mg/kg) and FTY720 than in pathological control group (** p≤0.01; *p≤0.05). FIG. 1 (C) shows clinical score (CS) per day study EAE-C06, where five different concentrations of BN201 (12.5 mg/kg, 25 mg/kg, 50 mg/kg, 100 mg/kg, and 150 mg/kg) were tested. Also a sham, a pathological control and FTY720 (at 2 mg/kg) comparator groups were included in the experiment. All groups were administered daily. Significant amelioration of the clinical (CS) score was observed for animals in the BN201 50 mg/kg, BN201 100 mg/kg and FTY720 2 mg/kg for almost all days of observation/treatment (Day 2 to day 30) (** p≤0.01; *p≤0.05).

FIG. 2, related with Example 2, is a plot of clinical score (CS) per day of EAE mice treated with Fingolimod (triangles), BN201 (light circles), combination of fingolimod and BN201 (dark circles), and placebo (squares). Arrow shows day of initiation of treatment. X-axis is the days after immunization of C57BL/6 mice to develop an EAE phenotype. p<0.05 differences between combination therapy (Fingolimod 0.1 mg/kg+ BN201 25 mg/kg) vs BN201 (25 mg/kg).

FIG. 3, related with Example 3, is a plot of clinical score (CS) per day of EAE mice treated with dimethyl-fumarate (DMF) (solid squares), BN201 (solid diamonds), combination of DMF and BN201 (cross), and placebo (triangles). X-axis is the days after immunization of C57BL/6 mice to develop an EAE phenotype.

FIG. 4, related with Example 4, is a plot of clinical score (CS) per day of EAE mice treated with STAT3 inhibitor S31-201 (cross), BN201 (triangles), combination of S31-201 and BN201 (squares), and placebo (circles). Arrow in shows day of initiation of treatment. X-axis is the days after immunization of C57BL/6 mice to develop an EAE phenotype.

FIG. 5, related with Example 6, is a graph with columns showing the viability percentage of human neuroblastoma cell line SH-SY5Y after oxidative stress conditions. Viability of cells treated with BN201 (first column), BN201-monomethylfumarate (second column) or Monomethyl Fumarate (third column) at different assay concentrations in the cell culture (0.03 μM, 0.1 μM, 0.3 μM, 0.5 μM, 1 μM, 3 μM, 5 μM, 10 μM, 20 μM, and 40 μM) are depicted in relation with a control (non-treated human neuroblastoma cells). The FIG. 5 shows that the cell viability, as percentage respect to control, is increased after pretreatment with BN201-monomethylFumarate salt respect to BN201 or Monomethyl Fumarate at the same tested concentration (*<0,0001 respect to BN201 only; #p<0.05 or ##p<0,0001 respect to Fumarate only).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

### Compounds of Formula (I)

As mentioned above, the present invention relates to a combination comprising:
a) a compound of formula (I), or a pharmaceutically or veterinary acceptable salt thereof,

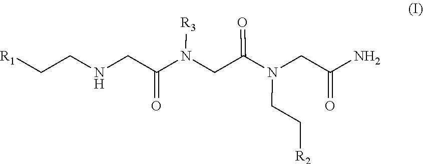

as previously defined, and
b) one or more drugs selected from the group consisting of: i) a compound of formula (IV) or a pharmaceutically or veterinary salt thereof, ii) a S1PR modulator, and iii) a STAT3 inhibitor.

In one embodiment, optionally in combination with one or more features of the various embodiments described above or below throughout all the description, in the compound of formula (I), R₁ is fluorophenyl, more particularly 2-fluorophenyl, 3-fluorophenyl or 4-fluorophenyl, even more particularly, 2-fluorophenyl.

In another embodiment, optionally in combination with one or more features of the various embodiments described above or below throughout all the description, in the compound of formula (I), R₁ is fluorophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is chlorophenyl, more particularly 2-chlorophenyl, 3-chlorophenyl or 4-chlorophenyl, even more particularly R₁ is 2-chlorophenyl.

In another embodiment, optionally in combination with one or more features of the various embodiments described above or below throughout all the description, in the compound of formula (I), R₁ is chlorophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is bromophenyl, more particularly 2-bromophenyl, 3-bromophenyl or 4-bromophenyl, and even more particularly 2-bromophenyl.

In another embodiment, optionally in combination with one or more features of the various embodiments described above or below throughout all the description, in the compound of formula (I), R₁ is bromophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is iodophenyl, more particularly 2-iodophenyl, 3-iodophenyl or 4-iodophenyl, and even more particularly 2-iodophenyl.

In another embodiment, optionally in combination with one or more features of the various embodiments described above or below throughout all the description, in the compound of formula (I), R₁ is iodophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is trifluoromethylphenyl, more particularly 2-trifluoromethylphenyl, 3-trifluoromethylphenyl or 4-trifluoromethylphenyl, and even more particularly 2-trifluoromethylphenyl.

In another embodiment, optionally in combination with one or more features of the various embodiments described above or below throughout all the description, in the compound of formula (I), R₁ is trifluoromethylphenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is pyrrolidin-1-yl.

In one embodiment, optionally in combination with one or more features of the various embodiments described above or below throughout all the description, in the compound of formula (I), R₂ is 2-oxo-pyrrolidin-1-yl-methyl.

In one embodiment, optionally in combination with one or more features of the various embodiments described above or below throughout all the description, in the compound of formula (I), R₂ is sulfamoylphenyl, more particularly 2-sulfamoylphenyl, 3-sulfamoylphenyl, or 4-sulfamoylphenyl, even more particularly 4-sulfamoylphenyl.

In one embodiment, optionally in combination with one or more features of the various embodiments described above or below throughout all the description, in the compound of formula (I), R₃ is 2-methylpropyl.

In one embodiment, optionally in combination with one or more features of the various embodiments described above or below throughout all the description, in the compound of formula (I), R₁ is 2-fluorophenyl or pyrrolidin-1-yl, and R₂ is 2-oxo-pyrrolidin-1-ylmethyl or 4-sulfamoylphenyl.

In another embodiment, optionally in combination with one or more features of the various embodiments described above or below throughout all the description, the compound of formula (I) of the combination of the invention as previously described, is selected from the group consisting of:

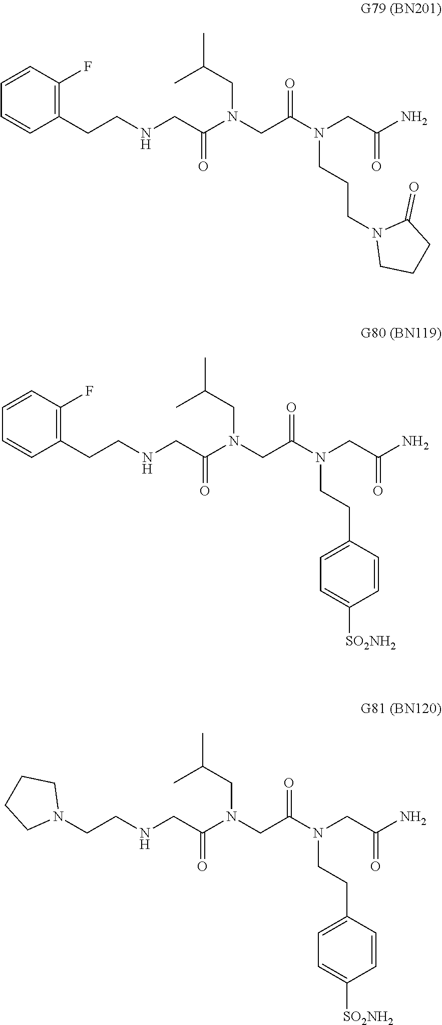 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

[N-(2-(2′-Fluorophenyl)ethyl)glycyl]-[N-(2-methylpropyl)glycyl]-N-[3-(2′-oxopyrrolidinyl)-propyl]glycinamide (G79, also known and labelled in Examples below as BN201). Chemical Formula: C₂₅H₃₈FN₅O₄; MW 491.5987.

[N-(2-(2′-Fluorophenyl)ethyl)glycyl]-[N-(2-methylpropyl)glycyl]-N-[2-(4′-sulfamoyl-phenyl)ethyl]glycinamide (G80, also known as BN119). Chemical Formula: C₂₆H₃₆FN₅O₅S; MW 549.658.

[N-(2-(1-Pyrrolidinyl)ethyl)glycyl]-[N-(2-methylpropyl)glycyl]-N-[2-(4′-Sulfamoyl-phenyl)ethyl]glycinamide (G81, also known as BN120). Chemical Formula: C₂₄H₄₀N₆OS; MW 524.6766.

Compounds of formula (I) and in particular G79 (BN201), G80 (BN119) and G81 (BN120), are members of a new class of peptoid non-chiral chemical compounds that function as agonists of trophic factors. Their synthesis and properties are disclosed in European Patent 2611775, “Agonists of neurotrophin receptors and their use as medicaments”. A notable feature of peptoid compounds is their lack of the amide hydrogen responsible for many of the secondary structural elements of peptides and proteins. One of these compounds, G79 (BN201), has been shown previously to exhibit neuroprotective effects in various multiple models of neuronal damage in vitro and in vivo. Other chemical names for BN201 include N-(2-amino-2-oxoethyl)-2-(2-((4-fluorophenethyl)amino)-N-isobutylacetamido-N-(3-(2-oxopyrrolidin-1-yl)propyl)acetamido, and N-({Carbamoylmethyl-[3-(2-oxo-pyrrolidin-1-yl)-propyl]-carbamoyl}-methyl)-2-[2-(2-fluoro-phenyl)-ethylamino]-N-isobutyl-acetamide.

There is no limitation on the type of salt of the compounds of formula (I) that can be used, provided that these are pharmaceutically or veterinary acceptable when they are used for therapeutic purposes. The term “pharmaceutically or veterinary acceptable salts”, embraces salts commonly used to form alkali metal salts and to form addition salts of free acids or free bases. The preparation of pharmaceutically or veterinary acceptable salts of the compounds of formula (I) can be carried out by methods known in the art. For instance, they can be prepared from the parent compound, which contains a basic or acidic moiety, by conventional chemical methods. Generally, such salts are, for example, prepared by reacting the free acid or base forms of the compounds of formula (I) with a stoichiometric amount of the appropriate pharmaceutically or veterinary acceptable base or acid in water or in an organic solvent or in a mixture of them. The compounds of formula (I) and their respective salts may differ in some physical properties but they are equivalent for the purposes of the present invention.

The compounds of the invention may be in crystalline form either as free solvation compounds or as solvates (e.g. hydrates) and it is intended that both forms are within the scope of the present invention. Methods of solvation are generally known within the art. In general, the solvated forms with pharmaceutically, cosmetically or veterinary acceptable solvents such as water, ethanol and the like are equivalent to the unsolvated form for the purposes of the invention.

In all embodiments of the invention referring to the compounds of formula (I), the pharmaceutically or veterinary acceptable salts thereof are always contemplated even if they are not specifically mentioned.

The compound of formula (I) (e.g., BN201) may be administered at a dosage from 0.5 mg/kg to 200 mg/kg in mice, that corresponds to a Human equivalent dose (HED) from 0.04 mg/kg to 16.26 mg/kg [HED calculation throughout this description based on Guidance for Industry estimating the maximum safe starting dose in initial clinical trials for therapeutics in adult healthy volunteers; FDA, CDER, July 2005]. Therapeutic or optimal doses are, in particular, of about 200 mg/kg or less, about 200 mg/kg, about 175 mg/kg or less, about 175 mg/kg, about 150 mg/kg or less, about 150 mg/kg, about 125 mg/kg or less, about 125 mg/kg, about 100 mg/kg or less, about 100 mg/kg, about 75 mg/kg or less, about 75 mg/kg, about 60 mg/kg or less, about 60 mg/kg, about 55 mg/kg or less, about 55 mg/kg, about 50 mg/kg or less, about 50 mg/kg, about 45 mg/kg or less, about 45 mg/kg, about 40 mg/kg or less, about 40 mg/kg, about 35 mg/kg or less, about 35 mg/kg, about 30 mg/kg or less, or about 30 mg/kg. Subtherapeutic or suboptimal doses are, in particular from 0.5 mg/kg to 25 mg/kg. In certain embodiments, the suboptimal dose of the compound of formula (I) (e.g., BN201), when in combination with one or more drugs as defined above, may be about 25 mg/kg or less, about 20 mg/kg or less, about 20 mg/kg, about 15 mg/kg or less, about 15 mg/kg, about 10 mg/kg or less, about 10 mg/kg, about 5 mg/kg or less, about 5 mg/kg, about 2.5 mg/kg or less, about 2.5 mg/kg, about 2.0 mg/kg or less, about 2.0 mg/kg, about 1.5 mg/kg or less, about 1.5 mg/kg, about 1.0 mg/kg or less, about 1.0 mg/kg, about 0.5 mg/kg or less, or about 0.5 mg/kg compound of formula (I) (e.g., BN201). In certain embodiments, administration of compound of formula (I) (e.g., BN201) at a suboptimal dose in combination with one or more of a compound of formula (IV), or a pharmaceutically or veterinary acceptable salt thereof, an SP1R inhibitor (e.g., fingolimod) and a STAT3 inhibitor (e.g., S3I-201) results in decreased side effects compared to the side effects associated with administration of compound of formula (I) (e.g., BN201) at an optimal dosage for treatment alone (e.g., pain, seizures, ataxia, etc.).

### Compounds of Formula (IV) and Salts Thereof

Compounds of formula (IV) or pharmaceutically or veterinary acceptable salts thereof are compounds derived from fumaric acid. These compounds correspond to the following formula:

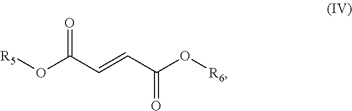

wherein R₅ is selected from hydrogen (H) and (C₁-C₆)alkyl, R₆ is selected from H, (C₁-C₆)alkyl and 2-(2,5-dioxopyrrolidin-1-yl)ethyl, and wherein if R₅ is H, R₆ is other than H.

In one embodiment, optionally in combination with one or more features of the various embodiments described above or below throughout all the description, in the compound of formula (IV), R₆ and R₅ are independently selected from hydrogen (H) and (C₁-C₆)alkyl, and wherein at least one of R₆ and R₅ is (C₁-C₆)alkyl. (C₁-C₆)alkyl relates to straight and branched alkyl groups selected from methyl, ethyl, propyl, isopropyl, butyl, isobutyl and tert-butyl, n-pentyl, isopentyl, neopentyl, n-hexyl, isohexyl and neohexyl.

In one embodiment, optionally in combination with one or more features of the various embodiments described above or below throughout all the description, in the compound of formula (IV), R₅ and R₆ are independently (C₁-C₄)alkyl; or R₅ is (C₁-C₄)alkyl and R₆ is hydrogen (H); being (C₁-C₄)alkyl in more in particular embodiment selected from the group consisting of methyl, ethyl, propyl, isopropyl, butyl, isobutyl and tert-butyl, when one or both of R₅ and R₆ are (C₁-C₄)alkyl.

In one embodiment, optionally in combination with one or more features of the various embodiments described above or below throughout all the description, in the compound of formula (IV), R₅ and R₆ are independently (C₁-C₄)alkyl; more in particular are selected from the group consisting of methyl, ethyl, propyl, isopropyl, butyl, isobutyl and tert-butyl.

In another particular embodiment optionally in combination with one or more features of the various embodiments described above or below throughout all the description, in the compound of formula (IV), R₅ and R₆ are the same (C₁-C₄)alkyl; more in particular are both methyl (dimethyl fumarate). Additional information regarding dimethyl fumarate is exposed below.

In one embodiment, optionally in combination with one or more features of the various embodiments described above or below throughout all the description, in the compound of formula (IV), R₅ is (C₁-C₄)alkyl and R₆ is hydrogen (H); more in particular R₅ is ethyl and R₆ is hydrogen (H) (monoethyl fumarate). In another more particular embodiment R₅ is methyl and R₆ is hydrogen (H) (monomethyl fumarate).

In one embodiment, optionally in combination with one or more features of the various embodiments described above or below throughout all the description, in the compound of formula (IV), R₆ is 2-(2,5-dioxopyrrolidin-1-yl)ethyl, and R₅(C₁-C₆)alkyl, more in particular R₅ is (C₁-C₄)alkyl, and even more in particular is methyl. When R₆ is 2-(2,5-dioxopyrrolidin-1-yl)ethyl and R₅ is methyl, the compound is also known as diroximel fumarate.

In also another embodiment, the pharmaceutically or veterinary salt of the compound of formula (IV) is a salt of a metal selected from the group of a metal of the Group I and a metal of the Group VIII. More in particular, it is a salt of sodium or a salt of iron (II) of the compound of formula (IV).

Inventors realized that besides compounds of formula (IV), salts of fumaric acid (HO₂CCH═CHCO₂H) of a metal selected from the group of a metal of the Group I and a metal of the Group VIII can also be used in combination with the compounds of formula (I) and optionally with any of a sphingosine-1-phosphate receptor modulator (S1PR modulator) and a Signal transducer and activator of transcription 3 (STAT3) inhibitor. In a particular embodiment, the salts are selected from sodium fumarate and iron fumarate.

All the particular embodiments of the compounds of formula (IV) in this description and aspects wherein it is included, also apply to these salts of fumaric acid of a metal of the Group I and a metal of the Group VIII.

Thus, it is also disclosed a combination comprising:
a) a compound of formula (I) or a pharmaceutically or veterinary acceptable salt thereof

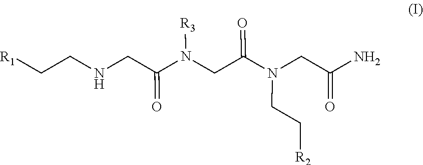

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl; and
b) one or more drugs selected from the group consisting of:
i) a compound of formula (IV), or a pharmaceutically or veterinary acceptable salt thereof

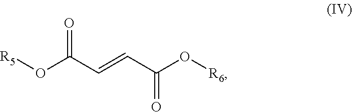

wherein R₅ is selected from hydrogen (H) and (C₁-C₆)alkyl, R₆ is selected from H, (C₁-C₆)alkyl and 2-(2,5-dioxopyrrolidin-1-yl)ethyl, and wherein if R₅ is H, R₆ is other than H,
ii) a sphingosine-1-phosphate receptor modulator (S1PR modulator),
iii) a Signal transducer and activator of transcription 3 (STAT3) inhibitor, and
iv) a salt of fumaric acid of a metal selected from the group of a metal of the Group I and a metal of the Group VIII.

In particular, the combination comprises: a) a compound of formula (I) or a pharmaceutically or veterinary acceptable salt thereof, and b) a salt of fumaric acid of a metal selected from the group of a metal of the Group I and a metal of the Group VIII.

Inventors also realized that a pharmaceutical preparation (i.e. capsule, pill, tablet) comprising fumaric acid or compounds of formula (IV) as disclosed above, or any pharmaceutically o veterinary acceptable salts of any of the acid or the compound of formula (IV), in combination with other active ingredients, in particular in combination with aspirin (i.e. acetylsalicylic acid) can also be used together with any of the compounds of formula (I) and optionally with one or more of a sphingosine-1-phosphate receptor modulator (S1PR modulator) and a Signal transducer and activator of transcription 3 (STAT3) inhibitor.

All the particular embodiments of the compounds of formula (IV) in this description also apply to these pharmaceutical preparations including in combination fumaric acid or compounds of formula (IV) or any pharmaceutically or veterinary acceptable salt thereof, and other active ingredients, in particular in combination with aspirin.

### Dimethyl Fumarate

Dimethyl fumarate (i.e., Tecfidera®), a dimethyl ester of fumarate, is approved in the United States and European Union for the treatment of adults with relapsing forms of MS. Dimethyl fumarate has been shown to possess immunomodulatory activity and anti-oxidant effects by, among other pathways, activating the nuclear factor (erythroid-derived 2)-like (Nrf2) pathway or GAPDH (see Tecfidera.com). However, the precise mechanism by which dimethyl fumarate works to fight relapsing MS is unknown. Dimethyl fumarate has the structure:

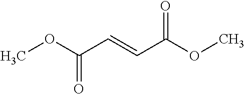

Dimethyl fumarate is generically used at a dosage at which it is capable of generating immunomodulatory activity when administered alone. The approved dosage of dimethyl fumarate is 120 mg twice daily for one week (equivalent to 1.7 mg/kg twice daily for a patient weighting 70 kg) and then about 240 mg twice daily thereafter (equivalent to 3.4 mg/kg twice daily for a patient weighting 70 kg). In certain embodiments, fumarate (e.g., dimethyl fumarate) may be for use as above indicated and administered in combination with a compound of formula (I) (e.g., BN201) at these dosages, known as therapeutic or optimal doses. Subtherapeutic dose of dimethyl fumarate are doses of less than 120 mg twice daily for one week and less than about 240 mg twice daily thereafter, less than about 120 mg twice per day, or less than about 120 mg once per day. In certain of these embodiments, administration of dimethyl fumarate may result in decreased side effects compared to side effects associated with administration dimethyl fumarate at an optimal dosage for treatment alone. In certain embodiments, the side effects from treatment with dimethyl fumarate that may be decreased as a result of administering dimethyl fumarate at a dosage that is lower than would normally be administered alone include, without limitation, anaphylaxis and angioedema, progressive multifocal leukoencephalopathy (PML), lymphopenia (i.e., decreased lymphocyte counts), flushing (i.e., sensation of heat or itching and a blush on the skin), gastrointestinal reactions (i.e., abdominal pain, diarrhea, and nausea), protein in the urine, elevated liver enzymes, rash, or some combination thereof.

Dimethyl fumarate can also be used in form of the delayed-release preparation (capsule) comprising in combination dimethyl fumarate and aspirin (i.e. VTS-72, a proprietary combination of Vitalis Pharma). This combination is proposed for the treatment of relapsing MS patients who experience fumarate flush.

### Monomethyl Fumarate

Monomethyl fumarate (i.e. Bafiertam®), also known as fumaric acid monomethyl ester, is indicated for the treatment of relapsing forms of multiple sclerosis (MS), to include clinically isolated syndrome, relapsing-remitting disease, and active secondary progressive disease, in adults. The approved dosage by FDA of monomethyl fumarate is of 95 mg twice (delayed-release capsules) a day, orally, for 7 days. Maintenance dose after 7 days is of 190 mg (administered as two 95 mg capsules) twice a day, orally.

### Diroximel Fumarate

Diroximel fumarate (i.e. Vumerity®), also know as 2-(2,5-Dioxopyrrolidin-1-yl)ethyl methyl fumarate, is indicated for the treatment of relapsing forms of multiple sclerosis (MS) in adults; specifically active secondary progressive disease and clinically isolated syndrome, as well as relapsing-remitting MS. Diroximel fumarate has the structure:

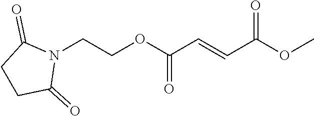 [SMILES: COC(=O)/C=C/C(=O)OCCN1C(=O)CCC1=O]

The approved dosage of diroximel fumarate is 231 mg twice a day, orally, for 7 days. Maintenance dose after 7 days is of 462 mg (administered as two 231 mg capsules) twice a day, orally.

Skilled person in the art is able to calculate doses of particular compound of formula (IV) with their own particular knowledge and considering guidance for the effect disclosed above.

### S1P Receptor (SPIR) Modulator

For the purposes of the present invention an “S1P receptor modulator” include compounds or agents which are able to decrease lymphocyte trafficking, modulating so the extent of an autoimmune attack. Lymphocyte trafficking is indirectly measured as lymphocytes in blood using flow cytometry. To be considered under S1PR modulator, lymphocyte trafficking is carried out by observation of the percentage of decrease or increase of lymphocytes in respect to baseline (before treatment with the SPIR modulator, i.e. Fingolimod). In a particular embodiment, S1PR modulators to be in the combination with a compound selected of formula (I) as defined above, are selected from the group consisting of fingolimod, siponimod, ozanimod, ponesimod and ceralifimod. (Patrick Vermersch; “Sphingosine-1-phosphate Receptor Modulators in Multiple Sclerosis”; European Neurological Review-2018; 13(1):25-30).

Fingolimod is a structural analogue of sphingosine derived from myriocin. Fingolimod (Gilenya®, Novartis; also referred to as FTY720) has been shown to possess immunomodulatory activity and is approved in the United States and European Union for reducing relapses and delaying disability progression in subjects with relapsing forms of MS. Although the precise mechanism by which fingolimod works to reduce relapses is unknown, it is thought that fingolimod decreases circulating lymphocytes by sequestering them in lymph nodes, which prevents them from contributing to an autoimmune reaction and attacking myelin (see Gilenya.com). Fingolimod has the structure:

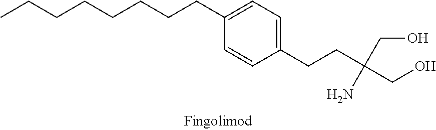 [SMILES: CCCCCCCCc1ccc(CCC(N)(CO)CO)cc1]

For SP1R modulators (e.g., fingolimod), therapeutic or optimal doses are those capable of generating immunomodulatory activity. The approved dosage of fingolimod is 0.5 mg/day for humans. In clinical trials, dosages of 1.25 mg/day and 5 mg/day fingolimod were shown to be the most effective dosages; however, side effects from fingolimod prevented its use at these doses. Particular therapeutic or optimal doses of fingolimod are from 0.75 mg/day to 50 mg/day. More in particular therapeutic doses are about 50 mg/day or less, about 50 mg/day, about 45 mg/day or less, about 45 mg/day, about 40 mg/day or less, about 40 mg/day, about 35 mg/day or less, about 35 mg/day, about 30 mg/day or less, about 30 mg/day, about 25 mg/day or less, about 25 mg/day, about 20 mg/day or less, about 20 mg/day, about 15 mg/day or less, about 15 mg/day, about 10 mg/day or less, about 10 mg/day, about 5 mg/day or less, about 5 mg/day, about 2.5 mg/day or less, about 2.5 mg/day, about 2.0 mg/day or less, about 2.0 mg/day, about 1.5 mg/day or less, about 1.5 mg/day, about 1.25 mg/day or less, about 1.25 mg/day, about 1.0 mg/day or less, about 1.0 mg/day, about 0.75 mg/day or less, or about 0.75 mg/day. As stated previously, the approved dosage of fingolimod is 0.5 mg/day.

Skilled person in the art is able to calculate doses of particular S1PR modulator with their own particular knowledge and considering guidance for the effect disclosed above.

Subtherapeutic or suboptimal doses of fingolimod (example of S1PR modulator) may be from 0.05 mg/day to 0.4 mg/day. In certain embodiments, fingolimod may be administered at a suboptimal dosage of 0.4 mg/day or less, about 0.3 mg/day or less, about 0.3 mg/day, about 0.2 mg/day or less, about 0.2 mg/day, about 0.1 mg/day or less, about 0.1 mg/day, about 0.05 mg/day or less, or about 0.05 mg/day. In certain of these embodiments, administration of fingolimod may result in decreased side effects compared to the side effects associated with administration of fingolimod at an optimal dosage for treatment alone. In certain embodiments, the side effects from treatment with fingolimod that may be decreased as a result of administering fingolimod at a dosage that is lower than would normally be administered alone include, without limitation, headache, flu, diarrhea, back pain, abnormal liver tests, cough, slow heart rate, increased risk of serious infections, posterior reversible encephalopathy syndrome (PRES), macular edema, swelling and narrowing of the blood vessels in the brain that may lead to a stroke or bleeding, breathing problems, macular edema, bradycardia, zoster reactivation, hemophagocytic syndrome, or some combination thereof.

Siponimod, marketed under the trade name Mayzent (FDA approval March 2019), is a selective sphingosine-1-phosphate receptor modulator (S1P₁ and S1P₅) for oral use that is used for multiple sclerosis (MS). It is intended for once-daily oral administration. Siponimod inhibits the migration of the lymphocytes to the location of the inflammation (e.g. in MS). It may be very similar to fingolimod but preventing lymphopenia, one of its main side effects.

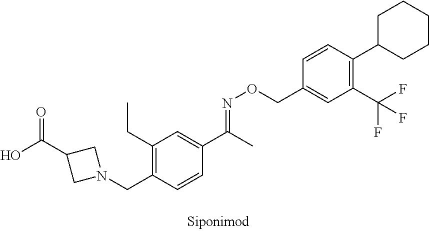

Ozanimod (RPC-1063) is an investigational immunomodulatory drug currently in phase III clinical trials for the therapy of relapsing multiple sclerosis (RMS) and ulcerative colitis (UC).

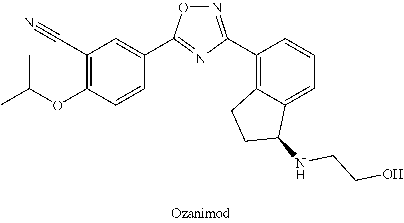

Ponesimod (INN, codenamed ACT-128800) is an experimental drug for the treatment of multiple sclerosis (MS) and psoriasis. It is being developed by Actelion.

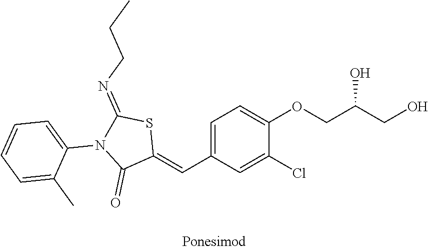

Ceralifimod (CAS No. 891859-12-4, or 1-[[6-[(2-methoxy-4-propylphenyl)methoxy]-1-methyl-3,4-dihydronaphthalen-2-yl]methyl]azetidine-3-carboxylic acid) interacts with the sphingosine-1-phosphate (S1P) receptors S1P₁ and S1P₅. Ceralifimod delays disease onset and inhibits lymphocyte infiltration of the spinal cord in a rat model of experimental autoimmune encephalomyelitis (EAE) and prevents disease relapse in a non-obese diabetic mouse model of relapsing-remitting EAE.

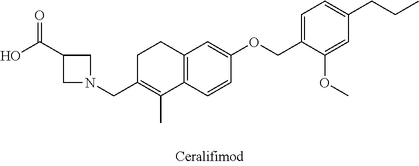

### STAT3 Inhibitors

For the purposes of the present invention as “STAT3 inhibitor” includes compounds or agents which are able to reduce activity of STAT3 in inducing signalling by pro-inflammatory cytokines. STAT3 inhibition may be measured by suppression of production of inflammatory cytokines in vitro and in vivo. Suppression of pSTAT3 expression and IL-17 production in myelin-specific CD4 T cells in a dose-dependent manner inhibit IL-6 induced IL-17 production in myelin-specific CD4 T cells.

The signal transducer and activator of transcription 3 gene (STAT3) is a transcription factor involved in several pathways and cell types, including being a damage sensor for axons protecting against axonal transection, or synaptic recycling. At the immunological level STAT3 is key mediator of IL-10 and IL-6 signalling pathways participating both in the anti-inflammatory response mediated by regulatory T cells as well as in the pro-inflammatory response mediated by Th17 cells. Non limiting examples of STAT3 inhibitors include (S,E)-3-(6-Bromopyridin-2-yl)-2-cyano-N-(1-phenylethyl)acrylamide (STAT3 Inhibitor III, WP1066, CAS 857064-38-1), 4-((3-(Carboxymethylsulfanyl)-4-hydroxy-1-naphthyl)sulfamoyl)benzoic acid (STAT3 Inhibitor IX Cpd188—CAS 823828-18-8), STAT3 Inhibitor Peptide (Linear Formula: C₃₈H₆₃N₈O₁₃P or C₉₂H₁₅₇N₂₀O₂₄P, or C₉₂H₁₅₆N₂₀O₂₁, SEQ ID NO: 1), 6-Nitrobenzo[b]thiophene-1,1-dioxide (STAT3 Inhibitor V Stattic—CAS 19983-44-9), 2-Hydroxy-4-[[[[(4-methylphenyl)sulfonyl]oxy]acetyl]amino]-benzoic acid (STAT3 Inhibitor VI S3I-201-CAS 501919-59-1), Ethyl-1-(4-cyano-2,3,5,6-tetrafluorophenyl)-6,7,8-trifluoro-4-oxo-1,4-dihydroquinoline-3-carboxylate (STAT3 Inhibitor VII—CAS 1041438-68-9), 5,15-Diphenylporphyrin (STAT3 Inhibitor VIII 5,15-DPP—CAS 22112-89-6), PIAS3 (UniprotKB database accession number Q9Y6X2, version 2 of sequence of Dec. 7, 2004—v2), N-(5-(Furan-2-yl)-1,3,4-oxadiazol-2-yl)-2-phenylquinoline-4-carboxamide (STAT3 Inhibitor XI STX-0119), STAT3 Inhibitor XII SPI (SEQ ID NO: 2), N-(1′,2-Dihydroxy-1,2′-binaphthalen-4′-yl)-4-methoxybenzenesulfonamide, (CAS No.: 432001-19-9, F1113-0789, STAT3 Inhibitor XIII C188-9)

SEQ ID NO: 1 disclosed above is: H-Pro-Tyr-(PO₃H₂)-Leu-Lys-Thr-Lys-Ala-Ala-Val-Leu-Leu-Pro-Val-Leu-Leu-Ala-Ala-Pro-OH (also named STAT3 Inhibitor Peptide)

SEQ ID NO: 2 disclosed above is H₂N-FISKERERAILSTKPPGTFLLRFSESSK-CO₂H (also named STAT3 Inhibitor XII SPI)

In one embodiment, optionally in combination with one or more features of the various embodiments described above or below throughout all the description, the STAT3 inhibitors is S3I-201, which is an amidosalicylic acid compound that selectively inhibits STAT3 activation and STAT3 dependent transcription. The chemical name for S3I-201 is the one indicated above: 2-Hydroxy-4-[[[[(4-methylphenyl)sulfonyl]oxy]acetyl]amino]-benzoic acid. S3I-201 has the structure:

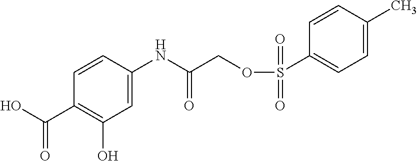 [SMILES: Cc1ccc(S(=O)(=O)OCC(=O)Nc2ccc(C(=O)O)c(O)c2)cc1]

Therapeutic doses of STAT3 inhibitors (e.g., S3I-201) are those capable of generating immunomodulatory activity when administered alone. Particular therapeutic or optimal doses are about 5 mg/kg. Subtherapeutic amounts are, thus, under these 5 mg/kg in mice. In particular, the subtherapeutic amount is from 0.1 to below 5 mg/kg in mice (that corresponds to HED of 0.01 mg/kg to below 0.4 mg/kg).

### Combinations of the Invention

As mentioned above, the present invention relates to a combination comprising:
a) a compound of formula (I), or a pharmaceutically or veterinary acceptable salt thereof, as previously defined, and
b) one or more drugs selected from the group consisting of: i) a compound of formula (IV) or a pharmaceutically or veterinary acceptable salt thereof, ii) a S1PR modulator, and iii) a STAT3 inhibitor, as previously defined.

In one embodiment, optionally in combination with one or more features of the various embodiments described above or below throughout all the description, the combination of the invention comprises:
a) a compound of formula (I), or a pharmaceutically or veterinary acceptable salt thereof, as previously defined, and
b) a drug selected from the group consisting of: i) a compound of formula (IV) or a pharmaceutically or veterinary acceptable salt thereof, ii) a S1PR modulator, and iii) a STAT3 inhibitor, as previously defined.

In another embodiment, optionally in combination with one or more features of the various embodiments described above or below throughout all the description, the combination of the invention comprises:
a) a compound of formula (I), or a pharmaceutically or veterinary acceptable salt thereof, as previously defined, and
b) one or more drugs selected from the group consisting of: i) a compound of formula (IV) or a pharmaceutically or veterinary acceptable salt thereof, ii) a SIPR modulator, and iii) a STAT3 inhibitor, as previously defined,
wherein the amount of a) and the amount of b) in combination are therapeutically effective.

In another embodiment, optionally in combination with one or more features of the various embodiments described above or below throughout all the description, in the combination of the invention the amount of a) separately is a subtherapeutic amount; and the amounts of a) and b) in combination are therapeutically effective.

In another embodiment, optionally in combination with one or more features of the various embodiments described above or below throughout all the description, in the combination of the invention the amount of b) separately is a subtherapeutic amount; and the amounts of a) and b) in combination are therapeutically effective.

In another embodiment, optionally in combination with one or more features of the various embodiments described above or below throughout all the description, in the combination of the invention the amount of a) and the amount of b) separately are subtherapeutic amounts; and the amounts of a) and b) in combination are therapeutically effective.

In one embodiment, optionally in combination with one or more features of the various embodiments described above or below throughout all the description, the combination of the invention comprises or consists of a) a compound of formula (I) as previously defined, which is in particular selected from the group consisting of compounds G79 (BN201), G80 (BN119) and G81 (BN120), even more in particular is G79 (BN201); and b) a compound of formula (IV) or a pharmaceutically or veterinary acceptable salt thereof, more in particular the compound of formula (IV) selected from dimethyl fumarate, monomethyl fumarate and monoethyl fumarate, even more in particular is dimethyl fumarate.

Other salts in combination with compound of formula (I) as previously defined, which is in particular selected from the group consisting of compounds G79 (BN201), G80 (BN119) and G81 (BN120), even more in particular is G79 (BN201) are the salts of fumaric acid disodium fumarate (sodium fumarate) and iron fumarate (iron (II) fumarate).

In another embodiment, optionally in combination with one or more features of the various embodiments described above or below throughout all the description, the combination of the invention comprises or consists of a) a compound of formula (I) as previously defined, which is in particular selected from the group consisting of compounds G79 (BN201), G80 (BN119) and G81 (BN120), even more in particular is G79 (BN201); and b) a S1PR modulator, which is in particular selected from the group consisting of fingolimod, siponimod and ozanimod, even more in particular is fingolimod.

In another embodiment, optionally in combination with one or more features of the various embodiments described above or below throughout all the description, the combination of the invention comprises or consists of a) a compound of formula (I) as previously defined, which is in particular selected from the group consisting of compounds G79 (BN201), G80 (BN119) and G81 (BN120), even more in particular is G79 (BN201); and b) a STAT3 inhibitor as defined herein, which is S3I-201.

### Other Drugs in Combination Meanwhile (a) and (b)

Other drugs for the treatment of multiple sclerosis are comprised, in other particular embodiments of the first aspect, in the combination comprising (a) a compound of formula (I), as defined above (i.e. including all possible pharmaceutically or veterinary acceptable salt thereof as previously indicated); and b) one or more drugs selected from the group consisting of a compound of formula (IV) or a pharmaceutically or veterinary acceptable salt thereof, a sphingosine-1-phosphate receptor modulator (S1PR modulator), and a Signal transducer and activator of transcription 3 (STAT3) inhibitor. These drugs include, in a more particular embodiment a drug selected from Interferon-beta, Glatiramer acetate, Natalizumab, Alentuzumab, Teriflunomide, Cladribine, Ocrelizumab and combinations thereof. Other drugs that could be combined are selected from Diroximel fumarate (ALKS 8700), Evobrutinib, Ofatumumab, Ublituximab, Amiloride, Fluoxetine, Ibudilast, Masitinib, MD1003 (Biotin), Opicinumab (Anti-LINGO-1, BIIB033), Riluzole, Simvastatin, Idebenone, Temelimab (GNbAC1), Inebilizumab (MEDI-551), naltrexone among others.

### Pharmaceutical and Veterinary Compositions, Package or Kit of Parts

The present invention also relates to pharmaceutical and veterinary compositions, or packages or kit of parts comprising a) a compound of formula (I) or a pharmaceutically or veterinary acceptable salt thereof, and b) one or more drugs selected from the group consisting of: i) a compound of formula (IV) or a pharmaceutically or veterinary acceptable salt thereof, ii) a S1PR modulator, and iii) a STAT3 inhibitor;

together with one or more pharmaceutically or veterinary acceptable excipients or carriers; wherein the compound of formula (I) and the drug are as defined above in any of the aspects and related particular embodiments.

The expression “pharmaceutically or veterinary acceptable excipients or carriers” refers to pharmaceutically or veterinary acceptable materials, compositions or vehicles. Each component must be pharmaceutically or veterinary acceptable in the sense of being compatible with the other ingredients of the pharmaceutical or veterinary composition. It must also be suitable for use in contact with the tissue or organ of humans and animals without excessive toxicity, irritation, allergic response, immunogenicity or other problems or complications commensurate with a reasonable benefit/risk ratio.

It forms part of the invention a single pharmaceutical or veterinary composition which comprises:
a) a compound of formula (I) or a pharmaceutically or veterinary acceptable salt thereof, and
b) one or more drugs selected from the group consisting of a compound of formula (IV) or a pharmaceutically or veterinary acceptable salt thereof, a S1PR modulator, and a STAT3 inhibitor;
together with one or more pharmaceutically or veterinary acceptable excipients or carriers; wherein the compound of formula (I) and the drugs are as defined previously, and wherein the amount of a) and the amount of b) in combination are therapeutically effective.

The expression “therapeutically effective” as used herein throughout this description, refers to the amount of a compound or combination of compounds that, when administered, is enough to prevent development of, or alleviate to some extent, one or more of the symptoms of the disease which is addressed. In this particular description, it is the amount of a compound, combination of compounds, or composition that produces a desired therapeutic effect in a subject, such as treating MS, NMO, and/or optic neuritis. The precise therapeutically effective amount is an amount of the composition that will yield the most effective results in terms of therapeutic efficacy in a given subject. The specific dose of the compound of the invention to obtain a therapeutic benefit may vary depending on the particular circumstances of the individual patient including, among others, the size, weight, age and sex of the patient, the nature and stage of the disease, the aggressiveness of the disease, and the route of administration. The specific dose of the compound of the invention to obtain a therapeutic benefit when administered in said combinations, compositions or kit of parts, may vary in relation with the specific dose of the compound used as single active agent.

Those particular embodiments of the combinations above, are also applicable as particular embodiments of the single pharmaceutical or veterinary compositions, packages or kits of parts of the invention.

Thus, in one embodiment, optionally in combination with one or more features of the various embodiments described above or below throughout all the description, in the single pharmaceutical or veterinary composition as defined above, the amount of a) separately is a subtherapeutic amount; and the amounts of a) and b) in combination are therapeutically effective.

In another embodiment, optionally in combination with one or more features of the various embodiments described above or below throughout all the description, in the single pharmaceutical or veterinary composition as defined above, the amount of b) separately is a subtherapeutic amount; and the amounts of a) and b) in combination are therapeutically effective.

In another embodiment, optionally in combination with one or more features of the various embodiments described above or below throughout all the description, in the single pharmaceutical or veterinary composition as defined above, the amount of a) and the amount of b) separately are subtherapeutic amounts; and the amounts of a) and b) in combination are therapeutically effective.

In one embodiment, optionally in combination with one or more features of the various embodiments described above or below throughout all the description, in the single pharmaceutical or veterinary composition as defined above, the amount of a) and the amount of b) both separately are therapeutic amounts; and the amounts of a) and b) in combination are therapeutically effective.

The term “subtherapeutic amount” or “suboptimal amount” as used throughout this description, as well as “subtherapeutic dose” or “suboptimal dose”, all terms interchangeably used as synonyms, is an amount/dose which is by itself non-therapeutically effective. Thus, a “suboptimal dose” or “subtherapeutic dose”, means a dose which is below the optimal (or therapeutic) dose (or dose ranges), approved for the health authorities as effective for that compound when used in single-compound therapy and for a particular addressed disease, such as MS, NMO, and/or optic neuritis. Thus, when the term “subtherapeutic amount” appears in the aspects or embodiments in this description, is to be understood as the amount of a particular compound below the therapeutic amount approved as effective by a health authority (i.e. European Medicines Agency of European Union, U.S. Food and Drug Administration, etc.) when used in single-compound therapy for a particular disease. As a mode of example, if the effective dose of BN201 (compound of formula I) in single-compound therapy is from higher than 25 mg/kg to 200 mg/Kg, suboptimal doses are from 0.5 mg/Kg to 25 mg/Kg. The approved dose of fingolimod (one SP1R inhibitor) is 0.5 mg/day in humans, being the most effective doses according to clinical trials of 1.25 mg/day and 5 mg/day. Suboptimal doses of SP1R inhibitor (i.e. fingolimod) are those lower than 0.5 mg/day, from 0.05 mg/day to 0.4 mg/day. In the same way, approved dosage of dimethyl fumarate is 120 mg twice daily for one week and then about 240 mg twice daily thereafter. Suboptimal doses of dimethyl fumarate are a dose less than about 120 mg twice daily for one week and less than about 240 mg twice daily thereafter. Therefore, in a particular embodiment the amount of a compound of formula (I) and the amount of the one or more of compounds selected from the group consisting of a compound of formula (IV) or a pharmaceutically or veterinary acceptable salt thereof, a S1PR modulator, and a STAT3 inhibitor when administered in said combinations, compositions or kit of parts is lower than an effective amount when used as single active agents.

The phrases “dose” and “dosage” are used interchangeably herein.

In another particular embodiment, optionally in combination with one or more features of the various embodiments described above or below throughout all the description, the invention also relates to a package or kit of parts comprising:
i) a first pharmaceutical or veterinary composition which comprises an amount of a compound of formula (I) as defined above, or a pharmaceutically or veterinary acceptable salt thereof, together with one or more pharmaceutically or veterinary acceptable excipients or carriers;
   ii) a second pharmaceutical or veterinary composition which comprises an amount of one or more drugs selected from the group consisting of a compound of formula (IV) or a pharmaceutically or veterinary acceptable salt thereof, a S1PR modulator, and a STAT3 inhibitor, which are as previously defined, together with one or more pharmaceutically or veterinary acceptable excipients or carriers; and
   iii) instructions for the use in combination of i) and ii);
wherein the first and second compositions are separate compositions, and wherein the amount of the compound of formula (I) of i) and the amount of one or more drugs of ii) in combination are therapeutically effective.

In one embodiment, optionally in combination with one or more features of the various embodiments described above or below throughout all the description, in the kit of parts as defined above, the amount of both the compound of formula (I) and the amount of one or more drugs of ii) separately are therapeutically amounts; and the amounts of the compound of formula (I) of i) and of the one or more drugs of ii) in combination are therapeutically effective.

In one embodiment, optionally in combination with one or more features of the various embodiments described above or below throughout all the description, in the kit of parts as defined above, the amount of the compound of formula (I) of i) separately is a subtherapeutic amount; and the amounts of the compound of formula (I) of i) and the amount of one or more drugs of ii) in combination are therapeutically effective.

In another embodiment, optionally in combination with one or more features of the various embodiments described above or below throughout all the description, in the kit of parts as defined above, the amount of one or more drugs of ii) separately is a subtherapeutic amount; and the amounts of the compound of formula (I) of i) and of the one or more drugs of ii) in combination are therapeutically effective.

In another embodiment, optionally in combination with one or more features of the various embodiments described above or below throughout all the description, in the kit of parts as defined above, the amount of the compound of formula (I) of i) and the amount of one or more drugs of ii) separately are subtherapeutic amounts; and the amounts of the compound of formula (I) of i) and of the one or more drugs of ii) in combination are therapeutically effective.

Particular embodiments of this kit of parts, optionally in combination with one or more features of the various embodiments described above or below throughout all the description, include cartridges with separate compartments including one of the pharmaceutical or veterinary composition; and the instructions for the use in combination of i) and ii), said instructions in particular in a form selected from a leaflet, a data carrier (i.e. CD, QR-code).

The election of the pharmaceutical or veterinary formulation will depend upon the nature of the active compound and its route of administration. Any route of administration may be used, for example oral, parenteral and topical administration.

For example, the pharmaceutical or veterinary composition may be formulated for oral administration and may contain one or more physiologically compatible carriers or excipients, in solid or liquid form. These preparations may contain conventional ingredients such as binding agents, fillers, lubricants, and acceptable wetting agents.

The pharmaceutical or veterinary composition may be formulated for parenteral administration in combination with conventional injectable liquid carriers, such as water or suitable alcohols. Conventional pharmaceutical or veterinary excipients for injection, such as stabilizing agents, solubilizing agents, and buffers, may be included in such compositions. These pharmaceutical or veterinary compositions may be injected intramuscularly, intraperitoneally, or intravenously.

The pharmaceutical composition may be formulated for topical administration. Formulations include creams, lotions, gels, powders, solutions and patches wherein the compound is dispersed or dissolved in suitable excipients.

The pharmaceutical compositions may be in any form, including, among others, tablets, pellets, capsules, aqueous or oily solutions, suspensions, emulsions, or dry powdered forms suitable for reconstitution with water or other suitable liquid medium before use, for immediate or retarded release.

The appropriate excipients and/or carriers, and their amounts, can readily be determined by those skilled in the art according to the type of formulation being prepared.

Treatment Inflammatory Neurological Diseases or Conditions which Result in the Destruction or Degeneration of Axons or Myelin

It forms part of the invention the combination, single pharmaceutical or veterinary composition, package or kit of parts comprising a) a compound of formula (I), or a pharmaceutically or veterinary acceptable salt thereof; and b) one or more drugs selected from the group consisting of i) a compound of formula (IV) or a pharmaceutically or veterinary acceptable salt thereof, ii) a S1PR modulator, and iii) a STAT3 inhibitor, wherein the compound of formula (I) and the drugs are as defined above for use in the treatment and/or prevention of an inflammatory neurological disease or condition which can result in the destruction or degeneration of axons or myelin in a subject in need thereof.

This aspect may also be formulated as a method of treatment and/or prevention of an inflammatory neurological disease or condition which can result in the destruction or degeneration of axons or myelin, which comprises administering to a mammal subject in need thereof, including a human subject, either
a) a therapeutically effective amount of the combination comprising (a) a compound of formula (I), or a pharmaceutically or veterinary acceptable salt thereof; and (b) one or more drugs selected from the group consisting of i) a compound of formula (IV) or a pharmaceutically or veterinary acceptable salt thereof, ii) a S1PR modulator, and iii) a STAT3 inhibitor; wherein the compound of formula (I) and the drugs are as defined above, together with one or more pharmaceutically or veterinary acceptable excipients or carriers; or alternatively
b) the package or kit of parts as defined in the embodiments above.

It also forms part of the invention the use of a combination comprising: (a) a compound of formula (I), or a pharmaceutically or veterinary acceptable salt thereof; and (b) one or more drugs selected from the group consisting of i) a compound of formula (IV) or a pharmaceutically or veterinary acceptable salt thereof, ii) a S1PR modulator, and iii) a STAT3 inhibitor, wherein the compound of formula (I) and the drugs are as defined above; for the preparation of a medicament for the treatment and/or prevention of an inflammatory neurological disease or condition which can result in the destruction or degeneration of axons or myelin.

In a particular embodiment, optionally in combination with one or more features of the various embodiments described above or below throughout all the description, the medicament comprises a single pharmaceutical or veterinary composition as defined in embodiment above or a package or kit of parts also as defined above.

In another particular embodiment of any of the single pharmaceutical or veterinary composition, or package or kit of parts, optionally in combination with one or more features of the various embodiments described above or below throughout all the description, the treatment comprises the simultaneous, concurrent, separate or sequential administration of (a) the compound of formula (I), or a pharmaceutically or veterinary acceptable salt thereof; and (b) one or more drugs selected from the group consisting of i) a compound of formula (IV) or a pharmaceutically or veterinary acceptable salt thereof, ii) a S1PR modulator, and iii) a STAT3 inhibitor, wherein the compound of formula (I) and the drugs are as defined above.

In one embodiment, optionally in combination with one or more features of the various embodiments described above or below throughout all the description, the combination, single pharmaceutical or veterinary composition, package or kit of parts for use as indicated above, comprises:
a) a compound of formula (I), or a pharmaceutically or veterinary acceptable salt thereof, as previously defined, and
b) a drug selected from the group consisting of: i) a compound of formula (IV) or a pharmaceutically or veterinary acceptable salt thereof, ii) a S1PR modulator, and iii) a STAT3 inhibitor, as previously defined.

In another embodiment, optionally in combination with one or more features of the various embodiments described above or below throughout all the description, the combination, single pharmaceutical or veterinary composition, package or kit of parts for use as indicated above, comprises:
a) a compound of formula (I), or a pharmaceutically or veterinary acceptable salt thereof, as previously defined, and
b) one or more drugs selected from the group consisting of: i) a compound of formula (IV) or a pharmaceutically or veterinary acceptable salt thereof, ii) a S1PR modulator, and iii) a STAT3 inhibitor, as previously defined,
wherein the amount of a) and the amount of b) in combination are therapeutically effective.

In another embodiment, optionally in combination with one or more features of the various embodiments described above or below throughout all the description, in the combination, single pharmaceutical or veterinary composition, package or kit of parts for use as indicated above, the amount of a) and the amount of b) separately are both therapeutic amounts; and the amounts of a) and b) in combination are therapeutically effective.

In another embodiment, optionally in combination with one or more features of the various embodiments described above or below throughout all the description, in the combination, single pharmaceutical or veterinary composition, package or kit of parts for use as indicated above, the amount of a) separately is a subtherapeutic amount; and the amounts of a) and b) in combination are therapeutically effective.

In another embodiment, optionally in combination with one or more features of the various embodiments described above or below throughout all the description, in the combination, single pharmaceutical or veterinary composition, package or kit of parts for use as indicated above, the amount of b) separately is a subtherapeutic amount; and the amounts of a) and b) in combination are therapeutically effective.

In another embodiment, optionally in combination with one or more features of the various embodiments described above or below throughout all the description, in the combination, single pharmaceutical or veterinary composition, package or kit of parts for use as indicated above, the amount of a) and the amount of b) separately are subtherapeutic amounts; and the amounts of a) and b) in combination are therapeutically effective.

In another embodiment, optionally in combination with one or more features of the various embodiments described above or below throughout all the description, in the combination, single pharmaceutical or veterinary composition, package or kit of parts for use as indicated above, the subtherapeutic amount of a) is from 0.5 mg/kg to 25 mg/kg (that corresponds to human equivalent dose (HED) of 0.04 mg/kg to 2 mg/kg).

In another embodiment, optionally in combination with one or more features of the various embodiments described above or below throughout all the description, in the combination, single pharmaceutical or veterinary composition, package or kit of parts for use as indicated above, b) is a compound of formula (IV) or a pharmaceutically or veterinary acceptable salt thereof, more in particular is dimethyl fumarate and the subtherapeutic amount of the compound of formula (IV) is of less than 120 mg twice daily for one week (equivalent to 1.7 mg/kg twice daily for a human patient weighting 70 kg) and less than about 240 mg twice daily thereafter (equivalent to 3.4 mg/kg twice daily for a human patient weighting 70 kg).

In another embodiment, optionally in combination with one or more features of the various embodiments described above or below throughout all the description, in the combination, single pharmaceutical or veterinary composition, package or kit of parts for use as indicated above, b) is an SP1R modulator and the subtherapeutic amount is from 0.05 mg/day to 0.4 mg/day in humans. More in particular, an amount from 0.05 mg/day to 0.1 mg/day.

In another embodiment, optionally in combination with one or more features of the various embodiments described above or below throughout all the description, in the combination, single pharmaceutical or veterinary composition, package or kit of parts for use as indicated above, b) is a STAT3 inhibitor and the subtherapeutic amount is from 0.1 to 5 mg/kg in mice (that corresponds to HED of 0.01 mg/kg to 0.4 mg/kg).

In one embodiment, optionally in combination with one or more features of the various embodiments described above or below throughout all the description, in the combination, single pharmaceutical or veterinary composition, package or kit of parts for use as indicated above, comprises or consists of a) a compound of formula (I) as previously defined, which is in particular selected from the group consisting of compounds G79 (BN201), G80 (BN119) and G81 (BN120), even more in particular is G79 (BN201); and b) a compound of formula (IV) or a pharmaceutically or veterinary acceptable salt thereof, which is in particular selected from dimethyl fumarate, monomethyl fumarate, and monoethyl fumarate, even more in particular is dimethyl fumarate.

Other salts in combination with compound of formula (I) as previously defined, which is in particular selected from the group consisting of compounds G79 (BN201), G80 (BN119) and G81 (BN120), even more in particular is G79 (BN201) are the fumaric acid salts disodium fumarate (sodium fumarate) and iron fumarate (iron (II) fumarate), that inventors realized that were active also in combination.

In another embodiment, optionally in combination with one or more features of the various embodiments described above or below throughout all the description, in the combination, single pharmaceutical or veterinary composition, package or kit of parts for use as indicated above, comprises or consists of a) a compound of formula (I) as previously defined, which is in particular selected from the group consisting of compounds G79 (BN201), G80 (BN119) and G81 (BN120), even more in particular is G79 (BN201); and b) a S1PR modulator, which is in particular selected from the group consisting of fingolimod, siponimod and ozanimod, even more in particular is fingolimod.

In another embodiment, optionally in combination with one or more features of the various embodiments described above or below throughout all the description, in the combination, single pharmaceutical or veterinary composition, package or kit of parts for use as indicated above, comprises or consists of a) a compound of formula (I) as previously defined, which is in particular selected from the group consisting of compounds G79 (BN201), G80 (BN119) and G81 (BN120), even more in particular is G79 (BN201); and b) a STAT3 inhibitor as defined herein, which is S3I-201.

In another embodiment, optionally in combination with one or more features of the various embodiments described above or below throughout all the description, in the combination, single pharmaceutical or veterinary composition, package or kit of parts for use as indicated above, comprises or consists of a) a compound of formula (I) as previously defined; which is in particular selected from the group consisting of compounds G79 (BN201), G80 (BN119) and G81 (BN120), even more in particular is G79 (BN201); and b) a STAT3 inhibitor as defined herein, in particular which is S3I-201, wherein the amount of a) and the amount of b) in combination are therapeutically effective, and are for use in chronic or advanced phase of the inflammatory neurological disease or condition which can result in the destruction or degeneration of axons or myelin, in particular a chronic or advanced phase of a disease or condition selected from the group consisting of multiple sclerosis (MS), neuromyelitis optica (NMO), optical neuritis, Balo disease, Schilder's disease, transverse myelitis, acute hemorrhagic leukoencephalitis, Marburg disease, or some combination thereof.

It also forms part of the invention a compound of formula (I), or a pharmaceutically or veterinary acceptable salt thereof, together with one or more pharmaceutically or veterinary acceptable excipients or carriers, for administration in combination with one or more drugs selected from the group consisting of i) a compound of formula (IV) or a pharmaceutically or veterinary acceptable salt thereof, ii) a S1PR modulator, and iii) a STAT3 inhibitor; together with one or more pharmaceutically or veterinary acceptable excipients or carriers, for simultaneous, concurrent, separate or sequential use in the treatment and/or prevention of an inflammatory neurological disease or condition which can result in the destruction or degeneration of axons or myelin in a subject in need thereof, wherein the compound of formula (I) and the one or more drugs are as defined previously.

It also forms part of the invention one or more drugs selected from the group consisting of i) a compound of formula (IV) or a pharmaceutically or veterinary acceptable salt thereof, ii) a S1PR modulator, and iii) a STAT3 inhibitor; together with one or more pharmaceutically or veterinary acceptable excipients or carriers, for administration in combination with a compound of formula (I), or a pharmaceutically or veterinary acceptable salt thereof, together with one or more pharmaceutically or veterinary acceptable excipients or carriers, for simultaneous, concurrent, separate or sequential use in the treatment and/or prevention of an inflammatory neurological disease or condition which can result in the destruction or degeneration of axons or myelin in a subject in need thereof, wherein the compound of formula (I) and the drug are as previously defined.

In another particular embodiment of the combination, the single pharmaceutical or veterinary composition, or the package or kit of parts for use as indicated above, the inflammatory neurological disease or condition which can result in the destruction or degeneration of axons or myelin is selected from the group consisting of multiple sclerosis (MS), neuromyelitis optica (NMO), optical neuritis, Balo disease, Schilder's disease, transverse myelitis, acute hemorrhagic leukoencephalitis, Marburg disease, or some combination thereof.

As used herein (see examples below), the terms “neuroprotection”, “neuroprotective”, or “neuroprotective effect” refer to the ability to prevent or reduce death or damage to nerve cells, including neurons and glia, or rescuing, resuscitating or reviving nerve cells and their extensions such as axons, dendrites and synapsis after damage, e.g., damage arising from or associated with pathological or harmful conditions in the brain, central nervous system or peripheral nervous system. Thus, this neuroprotective effect comprises the conferred ability of neuronal cells to maintain or recover their neuronal functions. The neuroprotective effect stabilizes the cell membrane of a neuronal cell or helps in the normalization of neuronal cell functions. It prevents the loss of viability or functions of neuronal cells. It comprises the inhibition of progressive deterioration of neurons that leads to cell death. It refers to any detectable protection of neurons from stress. Neuroprotection includes the regeneration of nerve cells and myelin, i.e. the re-growth of a population of nerve cells after disease or trauma.

Pharmaceutical compositions as described herein may be administered to a subject in need thereof from once or more times per day to once every month or once every several months.

Therapeutic or subtherapeutic amounts of each of the compounds of formula (I) and the one or more drugs as previously disclosed, can in addition be adjusted taking into account the weight of the subject that is going to receive the combinations, the single pharmaceutical or veterinary compositions disclosed above. The skilled man in the art will be able to determine such amounts considering the guidance in this description.

For the purposes of the invention, the term “treatment” or variants of the word means to reduce, stabilize, or inhibit the progression of inflammatory neurological diseases or conditions which can result in the destruction or degeneration of axons or myelin in patients already suffering from the disease. The term “prevention” is used herein to refer to include both preventing the onset of clinically evident inflammatory neurological diseases or conditions as above exposed and delaying its onset. Thus, the terms “treat,” “treating,” and “treatment” as used herein may refer to generating a complete or partial regression of the disease; eliminating, reducing, preventing, or delaying development of symptoms associated with the disease; preventing, delaying, or reducing the risk of the development or onset of the disease; preventing, delaying, or reducing the rate and/occurrence of relapses; preventing, delaying, or reducing the increased time to progression of disability; providing a neuroprotective effect; or some combination thereof. For example, treatment may refer to reducing accumulation of disability in a subject in need thereof. In certain embodiments, treatment may also refer to providing a neuroprotective effect, immunomodulatory response, or some combination thereof.

The phrases “patient” and “subject” are used interchangeably herein.

The term “about” as used herein means within 5% or 10% of a stated value or range of values.

### Salts of Formula (II): Fumarate Derivative Salts of Compounds of Formula (I)

The invention also relates to salts of formula (II), which are fumarate-derivative salts of particular compounds of formula (I):

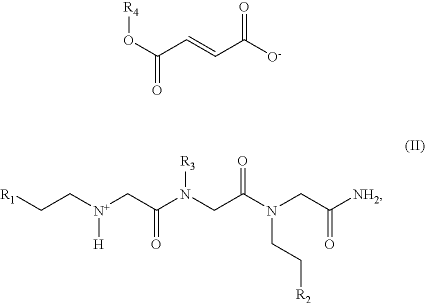

wherein R₁, R₂ and R₃ are as defined above for compounds of formula (I), and R₄ is (C₁-C₆)alkyl.

The preparation of the salt of formula (II) can be carried out by methods known in the art. For instance, they can be prepared from the parent compound of formula (I), which contains a basic moiety, by conventional chemical methods. Generally, such salts are, for example, prepared by reacting the free acid form of these compounds of formula (I) with a stoichiometric amount of the appropriate pharmaceutically or veterinary acceptable acid of formula (III) in water or in an organic solvent, such as methanol, ethanol or in a mixture of them, water and the organic solvent:

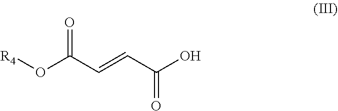

wherein R₄ is as defined above.

In one embodiment, optionally in combination with one or more features of the various embodiments described above or below throughout all the description, in the compound of formula (II), R₁ is fluorophenyl, more particularly 2-fluorophenyl, 3-fluorophenyl or 4-fluorophenyl, even more particularly, 2-fluorophenyl.

In another embodiment, optionally in combination with one or more features of the various embodiments described above or below throughout all the description, in the compound of formula (II), R₁ is fluorophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (II), R₁ is chlorophenyl, more particularly 2-chlorophenyl, 3-chlorophenyl or 4-chlorophenyl, even more particularly R₁ is 2-chlorophenyl.

In another embodiment, optionally in combination with one or more features of the various embodiments described above or below throughout all the description, in the compound of formula (II), R₁ is chlorophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (I), R₁ is bromophenyl, more particularly 2-bromophenyl, 3-bromophenyl or 4-bromophenyl, and even more particularly 2-bromophenyl.

In another embodiment, optionally in combination with one or more features of the various embodiments described above or below throughout all the description, in the compound of formula (II), R₁ is bromophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (II), R₁ is iodophenyl, more particularly 2-iodophenyl, 3-iodophenyl or 4-iodophenyl, and even more particularly 2-iodophenyl.

In another embodiment, optionally in combination with one or more features of the various embodiments described above or below throughout all the description, in the compound of formula (II), R₁ is iodophenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (II), R₁ is trifluoromethylphenyl, more particularly 2-trifluoromethylphenyl, 3-trifluoromethylphenyl or 4-trifluoromethylphenyl, and even more particularly 2-trifluoromethylphenyl.

In another embodiment, optionally in combination with one or more features of the various embodiments described above or below throughout all the description, in the compound of formula (II), R₁ is trifluoromethylphenyl which is further substituted with one or two substituents selected from the group consisting of halogen, (C₁-C₆)alkyl, (C₁-C₆)alkoxy, and halo(C₁-C₆)alkyl; preferably one or two substituents selected from the group consisting of halogen, (C₁-C₄)alkyl, (C₁-C₄)alkoxy, and halo(C₁-C₄)alkyl; more preferably one or two substituents selected from the group consisting of halogen, methyl, ethyl, propyl, isopropyl, methoxy, ethoxy, fluoromethyl, and trifluoromethyl.

In one embodiment, optionally in combination with one or more features of the various embodiments described above or below throughout all the description, in the compound of formula (II), R₁ is pyrrolidin-1-yl.

In one embodiment, optionally in combination with one or more features of the various embodiments described above or below throughout all the description, in the compound of formula (II), R₂ is 2-oxo-pyrrolidin-1-yl-methyl.

In one embodiment, optionally in combination with one or more features of the various embodiments described above or below throughout all the description, in the compound of formula (II), R₂ is sulfamoylphenyl, more particularly 2-sulfamoylphenyl, 3-sulfamoylphenyl, or 4-sulfamoylphenyl, even more particularly 4-sulfamoylphenyl.

In one embodiment, optionally in combination with one or more features of the various embodiments described above or below throughout all the description, in the compound of formula (II), R₃ is 2-methylpropyl.

In one embodiment, optionally in combination with one or more features of the various embodiments described above or below throughout all the description, in the compound of formula (II), R₁ is 2-fluorophenyl or pyrrolidin-1-yl, and R₂ is 2-oxo-pyrrolidin-1-ylmethyl or 4-sulfamoylphenyl.

In one embodiment, optionally in combination with one or more features of the various embodiments described above or below throughout all the description, in the compound of formula (II), and thus in compound of formula (III), R₄ is (C₁-C₄)alkyl, more preferably is selected from the group consisting of methyl, ethyl, propyl, isopropyl, butyl, isobutyl and tert-butyl.

In one embodiment, optionally in combination with one or more features of the various embodiments described above or below throughout all the description, in the compound of formula (II), and thus in compound of formula (III), R₄ is methyl.

In another embodiment, optionally in combination with one or more features of the various embodiments described above or below throughout all the description, the compound of formula (II) is

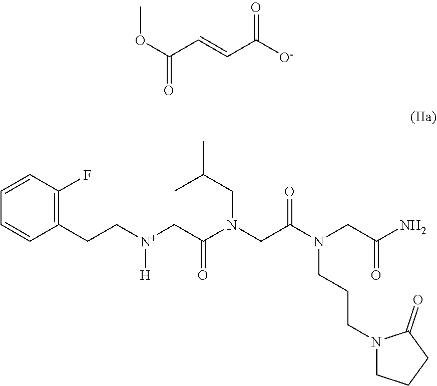

Particular salts of formula (II), more in particular fumarate salt of BN201 (called herewith BN201-fumarate salt, or compound of formula (IIa)) were generated (see Example 5) and evaluated for its neuroprotective effects versus BN201 or fumarate alone (see Example 6). Results showed that BN201 and fumarate both partially rescued neurons from death induced by oxidative stress. BN201-fumarate salt, however, exhibited a significantly higher level of protection than either compound alone, suggesting the presence of a synergistic neuroprotective effect.

Thus, all these salts of formula (II) may be added in pharmaceutical or veterinary compositions as active agents for preserving health of neurons and/or to rescue damaged neurons.

Therefore, the invention also relates to a pharmaceutical or veterinary composition comprising a therapeutically effective amount of the compound of formula (II), together with one or more pharmaceutically or veterinary acceptable excipients or carriers.

The invention also relates to the salt of formula (II), or to a pharmaceutical or veterinary composition comprising it, for use in the treatment and/or prevention of an inflammatory neurological disease or condition which can result in the destruction or degeneration of axons or myelin in a subject in need thereof.

This aspect may also be formulated as a method of treatment and/or prevention of an inflammatory neurological disease or condition which can result in the destruction or degeneration of axons or myelin, which comprises administering to a mammal subject in need thereof, including a human subject, a therapeutically effective amount of salt of formula (II), or a pharmaceutical or veterinary composition comprising it, together with one or more pharmaceutically or veterinary acceptable excipients or carriers.

It also forms part of the invention the use of salt of formula (II); for the preparation of a medicament for the treatment and/or prevention of an inflammatory neurological disease or condition which can result in the destruction or degeneration of axons or myelin.

In one embodiment, optionally in combination with one or more features of the various embodiments described above or below throughout all the description, the salt of formula (II), or a pharmaceutical or veterinary composition, or a kit of parts comprising it, for use as above disclosed, is for use in an inflammatory neurological disease or condition which can result in the destruction or degeneration of axons or myelin, selected from the group consisting of multiple sclerosis (MS), neuromyelitis optica (NMO), optical neuritis, Balo disease, Schilder's disease, transverse myelitis, acute hemorrhagic leukoencephalitis, Marburg disease, or some combination thereof.

In one embodiment, optionally in combination with one or more features of the various embodiments described above or below throughout all the description, the salt of formula (II), or a pharmaceutical or veterinary composition, is the salt of formula (IIa)

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1: Efficacy Pharmacology of BN201 (Example of Compound of Formula (I))

Three in vivo studies supporting the medical plausibility of BN201 in the treatment of MS and optic neuritis were performed. These studies sought to test the neuroprotective effect of BN201 and were conducted in the neurodegenerative and demyelinating experimental autoimmune encephalomyelitis (EAE) mouse model. Immune response to Myelin Basic Protein (MBP) or Myelin Proteolipid Protein (PLP) induces lesions located predominantly in the spinal cord whereas immunization with Myelin Oligodendrocyte Glycoprotein (MOG) generates lesions located predominantly in the optic nerve and spinal cord. In the literature, the EAE model is commonly used as a model of MS and optic neuritis, since it can cause irreversible visual loss (Kezuka et al, Analysis of the pathogenesis of experimental autoimmune optic neuritis. J Biomed Biotechnol. 2011; 2011:294046; Guo et al, Decreased neural stem/progenitor cell proliferation in mice with chronic/nonremitting experimental autoimmune encephalomyelitis. Neurosignals. 2010; 18(1):1-8).

Study EAE-C03: daily i.p. administration of BN201 at two different concentrations was tested versus placebo according to the following experimental design:
EAE affected placebo-treated animals (pathological control group);
EAE-affected animals treated with BN201 at 50 mg/kg dose and
EAE-affected animals treated with BN201 at 100 mg/kg dose.

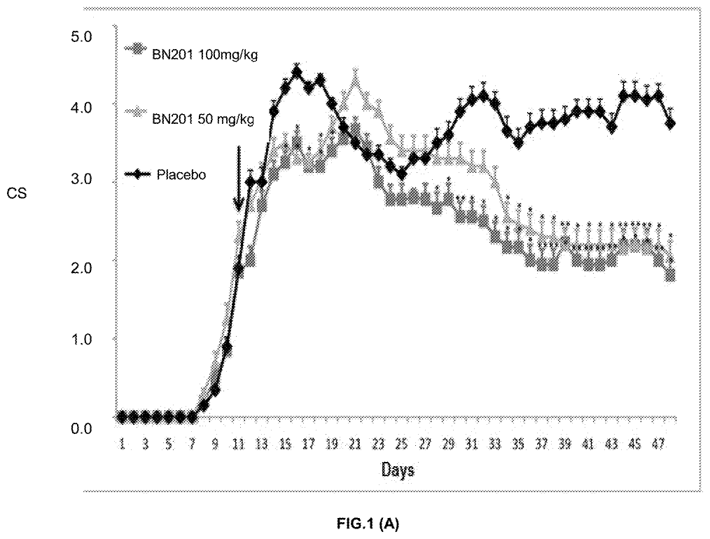

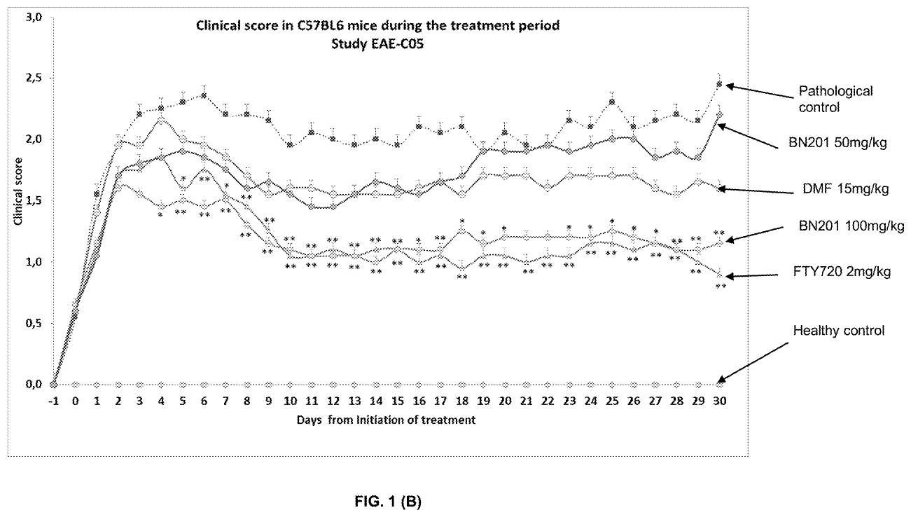

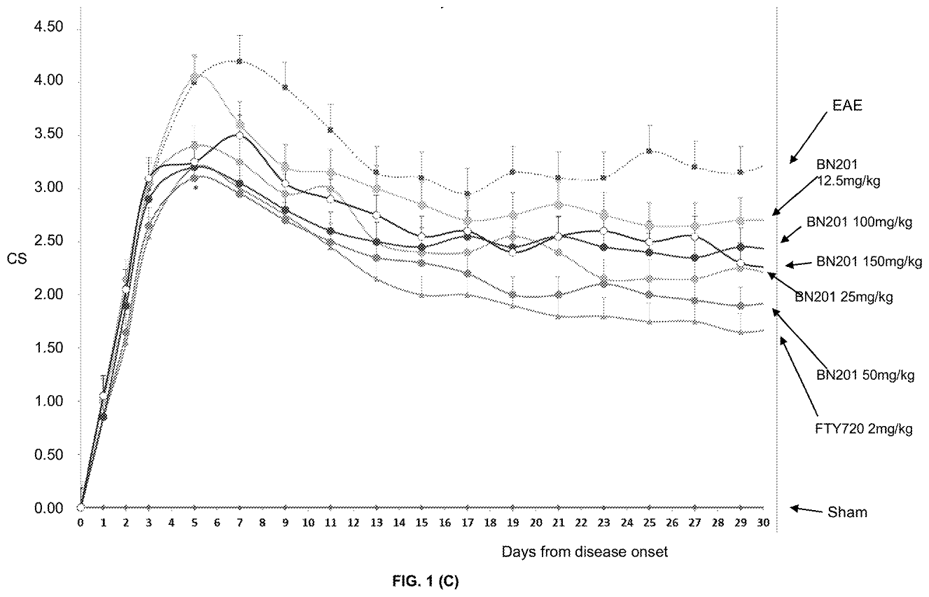

Treatment started in the chronic phase of the disease in order to test the curative properties of the molecule. Data are depicted in FIG. 1 (A), which is a graph plotting clinical score at day post-immunization for each of the tested groups (mean of the clinical score of the animals for each group). Results show that BN201 either at doses of 50 or 100 mg/kg significantly decreased the clinical score compared to placebo after day 17 days of treatment.

The EAE animal model is characterized by initial tail paralysis followed by hind limb paralysis and further progression to forelimb paralysis. Clinical scores were assigned to assess disease severity using the following scale: 0=normal; 0.5=mild limp tail; 1=limp tail; 2=mild paraparesis of the hind limbs, unsteady gait; 3=moderate paraparesis, voluntary movements still possible; 4=Severe paraparesis, almost complete hind limb paralysis; 5=Paraplegia o tetraparesis; 6=exitus.

Therapeutic treatment was initiated on day 11, once 70% of mice exhibited a clinical score of 2 or greater.

It is widely accepted that EAE is a complex model due to variability of incidence and severity of signs and symptoms between individuals. Thus, any effect of a tested drug is determined once the disease has started at the clinical level, thus some days after immunization and when behaviour in terms of clinical score is above a certain level (to be determined in the protocol) within all animals. Further meaningful data for a specific tested compound or protocol is achieved if observed parameter (e.g., clinical score) is maintained as meaningful between tested groups (assay, control, etc.) for a period of several days and between contiguous days within each group.

According to this interpretation code of the results, and applicable to all other figures in this description, in FIG. 1 (A), meaningful and conclusive data for EAE-affected animals treated with different doses of BN201, are the ones marked with an asterisk (*). P value of the statistical significance is defined in each figure footnotes or in the legend of figures. This set of marked data correspond to values of studied parameter (in this case the clinical score) in which animals in each group maintained the parameter in stabilized form between contiguous days, and as a whole data of the assayed group effectively differed from control (statistically difference among assayed groups; e.g., tested doses of compounds in relation with placebo, vehicle or sham animals).

A second experiment (Study EAE-C05) was then performed using the same mouse model.

Study EAE-C05: In addition to two dose levels of BN201 and a placebo-treated group (pathological control group), the study design introduced two active comparators, dimethyl fumarate (DMF) and fingolimod (FTY720), and a sham group (group in which animals were manipulated as the pathological group but without MOG injection). All groups were administered daily.
sham control non-diseased placebo-treated animals (healthy control group);
EAE-affected placebo-treated animals (pathological control group);
EAE-affected animals treated orally with 15 mg/kg of DMF;
EAE-affected animals treated with 2 mg/kg of FTY720;
EAE-affected animals treated with BN201 at 50 or 100 mg/kg.

Clinical scores were assigned to assess disease severity using the following scale: 0=normal; 0.5=Partial tail paralysis; 1=Complete tail paralysis; 1.5=Tail paralysis and loss of righting reflex; 2=Partial hind limb paralysis/paralysis of one limb; 2.5=One limb paralysis and partial loss of activity of one other limb; 3=Bilateral hind limb paralysis; 4=Moribund; 5=exitus.

Therapeutic treatment was initiated on day 12 or 13, once each mouse started showing complete tail paralysis (Score 1).

Data are depicted in FIG. 1 (B), which is a graph plotting clinical score (CS) per day from initiation of treatment, which shows clinical score for each of the tested groups (mean clinical score at each day for the animals within same group).

A third experiment (Study EAE-C06) to test dose response of BN201 was then performed using the same mouse model.

Study EAE-C06: five different concentrations of BN201: 12.5 mg/kg, 25 mg/kg, 50 mg/kg, 100 mg/kg, and 150 mg/kg. Also sham, pathological control and FTY720 (2 mg/kg) comparator groups were included in the experiment. All groups were administered daily.

Clinical scores were assigned to assess disease severity using the following scale: 0=normal; 0.5=Partial tail paralysis; 1=Complete tail paralysis; 1.5=Tail paralysis and loss of righting reflex; 2=Partial hind limb paralysis/paralysis of one limb; 2.5=One limb paralysis and partial loss of activity of one other limb; 3=Bilateral hind limb paralysis; 4=Moribund; 5=exitus.

Therapeutic treatment was initiated on day 12 once each mouse started showing complete tail paralysis (Score 1).

Data are depicted in FIG. 1 (C), wherein the clinical score for each of the tested groups is plotted per day after disease onset. Results shows a significant amelioration of the clinical score for animals in the BN201 50 mg/kg, BN201 100 mg/kg and FTY720 2 mg/kg for almost all days of observation/treatment (Day 2 to day 30) (TABLE 1).

TABLE 1

| | Comparison with EAE group-Clinical | | | | | |
|---|---|---|---|---|---|---|
| | scores (t-test p values) | | | | | |
| Days of | FTY720 | BN201 | BN201 | BN201 | BN201 | BN201 |
| treatment | 2 mpk | 12.5 mpk | 25 mpk | 50 mpk | 100 mpk | 150 mpk |
| 0 | ns | ns | ns | ns | ns | ns |
| 1 | ns | ns | ns | ns | ns | ns |
| 2 | ns | ns | ns | ns | ns | ns |
| 3 | ns | ns | ns | ns | ns | ns |
| 5 | * | ns | ns | * | ** | * |
| 7 | ** | * | ** | ** | ** | * |
| 9 | ** | ** | ** | ** | ** | ** |
| 11 | ** | ns | ns | ** | ** | ns |
| 13 | ** | ns | * | * | * | ns |
| 15 | ** | ns | * | * | * | ns |
| 17 | ** | ns | ns | * | ns | ns |
| 19 | ** | ns | ns | ** | * | * |
| 21 | ** | ns | ns | * | ns | ns |
| 23 | ** | ns | * | * | ns | ns |
| 25 | ** | * | ** | ** | ** | * |
| 27 | ** | * | ** | ** | ** | ns |
| 29 | ** | ns | ** | ** | * | ** |
| 31 | ** | ns | ** | ** | * | ** |

### Example 2. Comparison of the Combination of BN201 and Fingolimod Versus BN201 or Fingolimod Alone

Effect on the clinical progression of EAE in 8-12 week old female C57BL/6 mice was evaluated. Mice were administered once a day for six days per week at suboptimal dosages:
sham control non-diseased placebo-treated animals (healthy control group);
EAE-affected placebo-treated animals (pathological control group);
EAE-affected animals treated with 0.1 mg/kg of FTY720,
EAE-affected animals treated with 25 mg/kg of BN201, or
EAE-affected animals treated with a combination thereof.

The combination of BN201 and fingolimod was evaluated for its effect on the clinical progression of EAE in 8-12 weeks old female C57BL/6 mice. The EAE animal model is characterized by initial tail paralysis followed by hind limb paralysis and further progression to forelimb paralysis.

Clinical scores were assigned to assess disease severity using the following scale: 0=normal; 0.5=mild limp tail; 1=limp tail; 2=mild paraparesis of the hind limbs, unsteady gait; 3=moderate paraparesis, voluntary movements still possible; 4=Severe paraparesis, almost complete hind limb paralysis; 5=Paraplegia o tetraparesis; 6=exitus.

Acclimation, mice distribution based on weight and immunization was performed as in Example 1.

Starting on day 15, when 70% of the animals has a clinical score≥1, mice were administered Fingolimod, BN201, or a combination thereof once a day for six days per week at suboptimal dosages (0.1 mg/kg for Fingolimod, 25 mg/kg for BN201).

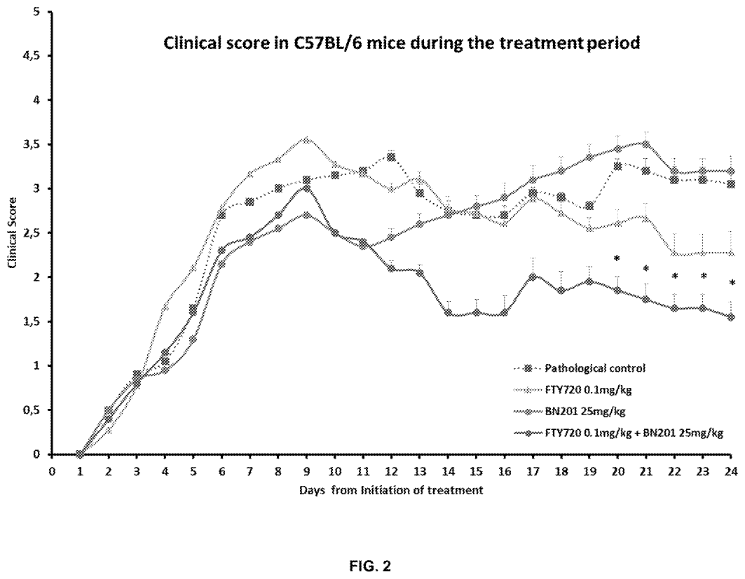

The results are summarized in FIG. 2. Mice administered a combination of BN201 and fingolimod exhibited a significantly greater improvement (p≤0.05) in the mean daily clinical scores from day 30 to day 35 than mice administered either compound alone. These differences were greater than the sum of the effect from each compound, suggesting doses much below the therapeutic ones for each of the compounds administered alone were effective when administered together.

### Example 3: Comparison of the Combination of BN201 and Dimethyl Fumarate (DMF) Versus BN201 or DMF Alone

Effect in the animal model of MS (i.e., EAE in C57BL6 mice immunized with MOG35-55), similar to Example 2.

Starting on day 15, mice were administered:
dimethyl fumarate (DMF) (group A, 10 mice),
BN201 (group B, 10 mice)
a combination thereof (group C, 10 mice)
   once a day for six days per week at suboptimal dosages (10 mg/kg for DMF, 25 mg/kg for BN201).

DMF was administered orally with a rigid cannula in a saline vehicle at a volume of 10 mL/kg, while BN201 was administered via i.p. injection in a saline vehicle at a volume of 5 mL/kg. Control mice received vehicle only via both oral and i.p. injection (group D, 10 mice) or nothing at all (group E, 2 mice).

The objective of the study was to evaluate the efficacy and safety of combination therapy with suboptimal doses of BN201 (25 mg/kg) and dimethyl-fumarate (DMF) (10 mg/kg) in the progression of chronic EAE in mice.

During a one week acclimation period starting on day −7, mice were distributed based on body weight stratification into different experimental groups. On day 0, mice were immunized subcutaneously in both hind pads with 150 μg of MOG peptide 35-55 (Spikem, Firenze) emulsified with 50 μg of Mycobacterium tuberculosis (H37Ra strain; Difco, Detroit, MI) in incomplete Freund's adjuvant (IFA). Mice were injected intraperitoneally (i.p.) with Pertussis toxin (Sigma) (500 ng) at the time of immunization and again two days later. Clinical scores were assigned to assess disease severity using the following scale: 0=normal; 0.5=mild limp tail; 1=limp tail; 2=mild parapesis of the hind limbs, unsteady gait; 3=moderate parapesis, voluntary movements still possible; 4=paraplegia or tetraparesis; 5=moribund state; 6=exitus. By day 14, 70% of mice exhibited a clinical score of 1 or greater.

Starting on day 15, mice were administered dimethyl fumarate (DMF) (group A, 10 mice), BN201 (group B, 10 mice), or a combination thereof (group C, 10 mice) once a day for six days per week at suboptimal dosages (10 mg/kg for DMF, 25 mg/kg for BN201). DMF was administered orally with a rigid cannula in a saline vehicle at a volume of 10 mL/kg, while BN201 was administered via i.p. injection in a saline vehicle at a volume of 5 mL/kg. Control mice received vehicle only via both oral and i.p. injection (group D, 10 mice) or nothing at all (group E, 2 mice). Animals were weighed and inspected for clinical signs of disease six days per week by a blinded observer. On day 30, mice were anesthetized and perfused intracardially with 4% paraformaldehyde in 0.1 M phosphate buffer (pH 7.6). Eyes, optic nerves, spinal cord, and brain were dissected and fixed until use.

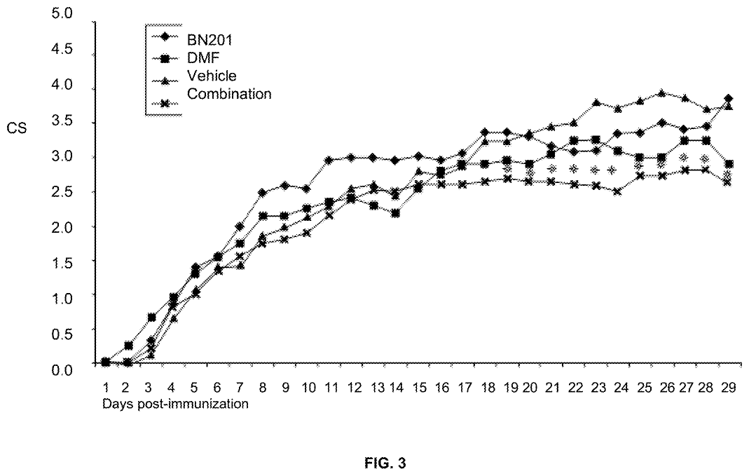

As shown in FIG. 3, animals treated with either BN201 or DMF at suboptimal doses suffered EAE similar to those mice treated with placebo. By contrast, the combination of suboptimal doses of BN201 in combination with DMF ameliorated the course of the disease in a significant manner from day 34 to 38 (see in FIG. 3 values with asterisks (*) indicate p<0.05). In summary, combination therapy of BN201 and DMF shows a synergistic effect for ameliorating the course of EAE.

### Example 4: Comparison of the Combination of BN201 and a STAT3-Inhibitor Versus BN201 or the STAT3 Inhibitor Alone

Effect during the chronic phase of the disease in the animal model of Multiple Sclerosis (i.e., EAE in C57BL6 mice immunized with MOG35-55)

Efficacy and safety of combination therapy with optimal doses of BN201 (50 mg/kg) and S31-201 (5 mg/kg) in the progression of chronic EAE in mice.

Starting the chronic phase of the disease (by day 34) mice were administered:
the STAT3 inhibitor S31-201 (group A, 7 mice),
BN201 (group B, 6 mice), or
a combination thereof (group C, 6 mice)
   once a day for six days per week at optimal dosages (5 mg/kg for S31-201, 50 mg/kg for BN201).

S31-201 and BN201 were administered via i.p. injection in a saline vehicle at a volume of 5 mL/kg. Control mice received vehicle only via both oral and i.p. injection (group D, 6 mice) or nothing at all (group E, 2 mice). Mice were treated from day 34 (chronic EAE stage) until the end of the experiment on day 54.

The combination of BN201 and the STAT3-inhibitor S31-201 was evaluated for its effect during the chronic phase of the disease in the animal model of Multiple Sclerosis (i.e., EAE in C57BL6 mice immunized with MOG35-55). The objective of the study was to evaluate the efficacy and safety of combination therapy with optimal doses of BN201 (50 mg/kg) and 531-201 (5 mg/kg) in the progression of chronic EAE in mice.

During a one week acclimation period starting on day −7, mice were distributed based on body weight stratification into different experimental groups. On day 0, mice were immunized subcutaneously in both hind pads with 150 μg of MOG peptide 35-55 (Spikem, Firenze) emulsified with 50 μg of Mycobacterium tuberculosis (H37Ra strain; Difco, Detroit, MI) in incomplete Freund's adjuvant (IFA). Mice were injected intraperitoneally (i.p.) with Pertussis toxin (Sigma) (500 ng) at the time of immunization and again two days later. Clinical scores were assigned to assess disease severity using the following scale: 0=normal; 0.5=mild limp tail; 1=limp tail; 2=mild parapesis of the hind limbs, unsteady gait; 3=moderate parapesis, voluntary movements still possible; 4=paraplegia or tetraparesis; 5=moribund state; 6=exitus. By day 17, more than 70% of mice exhibited a clinical score of 1 or greater.

Starting on day 34, mice were administered the STAT3 inhibitor S31-201 (group A, 7 mice, Sigma), BN201 (group B, 6 mice), or a combination thereof (group C, 6 mice) once a day for six days per week at optimal dosages (5 mg/kg for S31-201, 50 mg/kg for BN201). S31-201 and BN201 were administered via i.p. injection in a saline vehicle at a volume of 5 mL/kg. Control mice received vehicle only via both oral and i.p. injection (group D, 6 mice) or nothing at all (group E, 2 mice). Mice were treated from day 34 (chronic EAE stage) until the end of the experiment on day 54. Mice we randomized to each treatment at the beginning of the study. At the time of starting therapy, groups may have different levels of EAE severity (e.g BN201 alone group has more severe disease from onset of therapy than placebo). For this reason, comparison between groups was based in the change of the EAE score after therapy onset. Animals were weighed and inspected for clinical signs of disease six days per week by a blinded observer. On day 55, mice were anesthetized and perfused intracardially with 4% paraformaldehyde in 0.1M phosphate buffer (pH 7.6). Eyes, optic nerves, spinal cord, and brain were dissected and fixed until use.

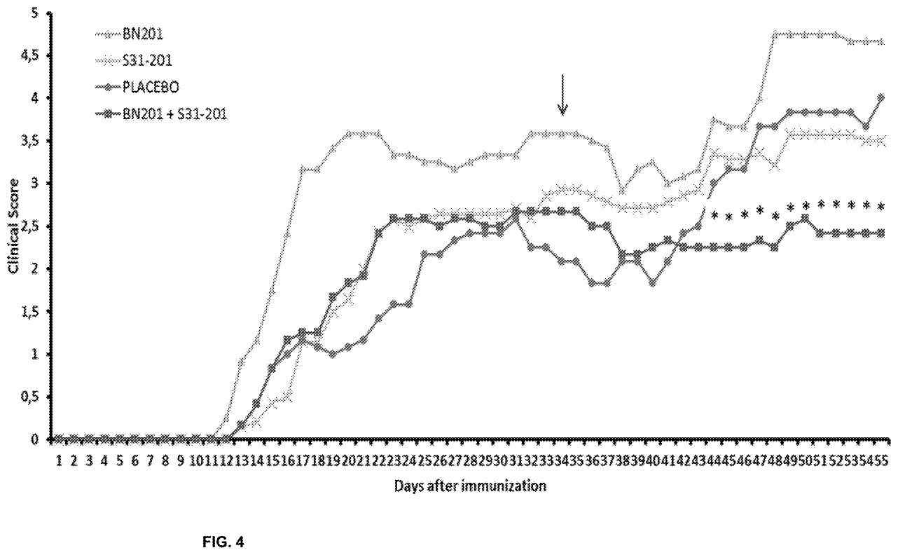

As shown in FIG. 4, treatment in the chronic phase of the disease (i.e., day 34, see arrow) with optimal doses of BN201 or optimal doses of S31-201 alone was not efficacious at this stage of the disease. However, treatment with optimal doses of the combination of BN201 and S31-201 ameliorated the course of EAE during the chronic phase of the disease in a significant manner. During the treatment, the clinical score was significantly lower in the combination therapy compared to the other treatments. The combination therapy of BN201 and STAT3 inhibitor S31-201 significantly protected mice suffering EAE in late chronic phases. Because the magnitude of the effect was higher than the sum of the effect of each drug in isolation, this suggests the presence of a synergistic activity between both drugs.

### Example 5. Preparation of a BN201-Monomethylfumarate Salt

For the preparation of the BN201-monomethylfumarate salt, 100 mg of AM-G79_03 (BN201) (1 eq) and 26 mg of mono-methyl fumarate (1 eq) were mixed in methanol and kept the mixture by stirring for 1 hour. After that concentration at vacuo was performed and the obtained residue was analyzed by ¹H-NMR.

Data of ¹H-NMR effectively corroborated that the compound of formula (IIa) below was obtained:

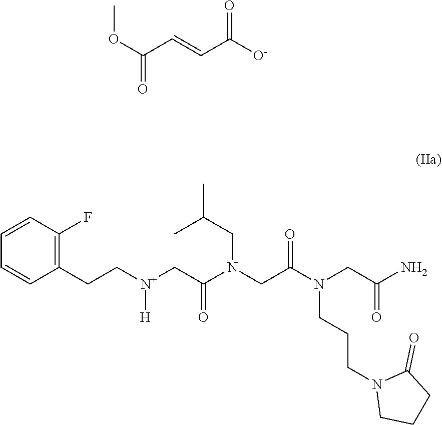

### Example 6. Comparison of BN201-Monomethylfumarate Salt Versus BN201 or Monomethyl Fumarate Alone

The objective of the study was to test the possible synergistic effect of the BN201-monomethylfumarate salt in a neuroprotective assay. Thus, BN201-monomethylfumarate salt obtained as indicated in example 5, was tested for its ability to protect the human neuroblastoma cell line SH-SY5Y against death induced by oxidative stress (i.e., hydrogen peroxide (H₂02)) using an MTT (3-(4,5-dimethylthiazol-2-yl)-2,5-diphenyltetrazolium bromide) cell proliferation assay.

SH-SY5Y cells were cultivated in 50% Eagle's minimum essential medium (EMEM), 50% Ham's F12 Nutrient Mixture, 10% fetal bovine serum (FBS), 2 mM L-Glu, and 1% penicillin/streptomycin. All of the cell cultures were maintained in a humidified incubator at 5% CO₂ and at 37° C. SH-SY5Y cells were preincubated for 1 hour with BN201 alone, monomethyl fumarate alone, or BN201-monomethylfumarate salt at various concentrations (n=5; concentrations: 0.03 μM, 0.1 μM, 0.3 μM, 0.5 μM, 1 μM, 3 μM, 5 μM, 10 μM, 20 μM, 40 μM) and then H₂O₂ (15 μM) was added to induce stress. Preincubation with sodium pyruvate (10 mM) was used as a positive control. After 30 minutes of H₂O₂ incubation, the medium was changed and thiazolyl blue tetrazolium bromide (MTT; Sigma Aldrich; stock concentration 10 mg/ml) was added to each well at the final concentration of 0.5 mg/ml. After 2 hours of MTT incubation the cell medium was removed, and cells were resuspended in pure dimethyl sulfoxide (DMSO). Cell viability was determined by reading the absorbance at 570 nm. Each experiment was performed in quintuplicate.

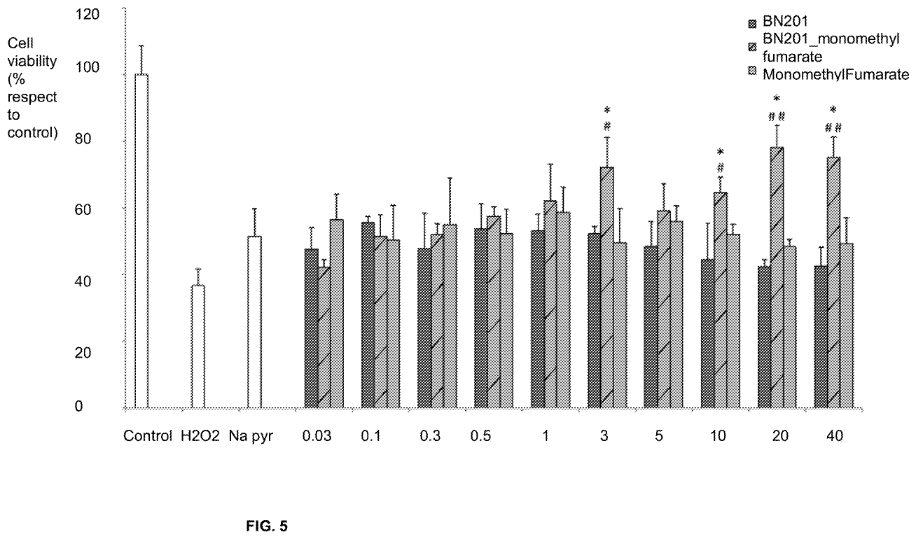

The results show that the cell viability, provided as a percentage compared to the control, is increased after pre-treatment with BN201-monomethylfumarate salt compared to BN201 or fumarate tested alone at the same concentration. Data are depicted in FIG. 5.

BN201 and fumarate both provided partially rescued neurons from death induced by oxidative stress, with an efficacy similar well-known anti-oxidants such as sodium pyruvate. BN201-monomethylfumarate, on the other hand, exhibited a significantly higher level of neuroprotection than either compound alone, which indicates that the BN201-monomethylfumarate salt exerts a synergistic neuroprotective effect.

Further aspects/embodiments of the present invention can be found in the following clauses:

Clause 1. A combination comprising:
a) a compound of formula (I) or a pharmaceutically or veterinary acceptable salt thereof

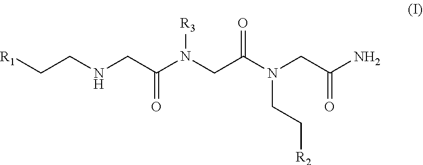

wherein:
R₁ is phenyl substituted with halogen or trifluoromethyl, and further optionally substituted with one or two substituents selected from the group consisting of halogen, (C₁-C₆)alkyl, (C₁-C₆)alkoxy, and halo(C₁-C₆)alkyl; or alternatively R₁ is pyrrolidin-1-yl;
R₂ is 2-oxo-pyrrolidin-1-ylmethyl or sulfamoylphenyl; and
R₃ is chosen from propyl, 1-methylethyl, butyl, 2-methylpropyl, pentyl, 1-methyl-butyl, 2-methylbutyl, hexyl, 4-methylpentyl, 3-methylpentyl, 2-methylpentyl, and 1-methylpentyl; and
b) one or more drugs selected from the group consisting of:
(i) a compound of formula (IV), or a pharmaceutically or veterinary acceptable salt thereof

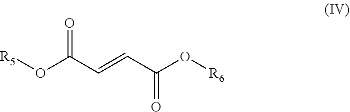

wherein R₆ and R₅ are independently selected from hydrogen (H) and (C₁-C₆)alkyl;
(ii) a sphingosine-1-phosphate receptor inhibitor (S1PR modulator); and
(iii) a Signal transducer and activator of transcription 3 (STAT3) inhibitor.

Clause 2. The combination according to clause 1, which comprises a compound of formula (I), and a drug selected from the group consisting of a compound of formula (IV), or a pharmaceutically or veterinary acceptable salt thereof, a S1PR modulator, and a STAT3 inhibitor.

Clause 3. The combination according to any of clauses 1-2, wherein in the compound of formula (I), R₃ is 2-methylpropyl and R₁ and R₂ are as defined in claim 1.

Clause 4. The combination according to clause 3, wherein the compound of formula (I) is selected from the group consisting of:

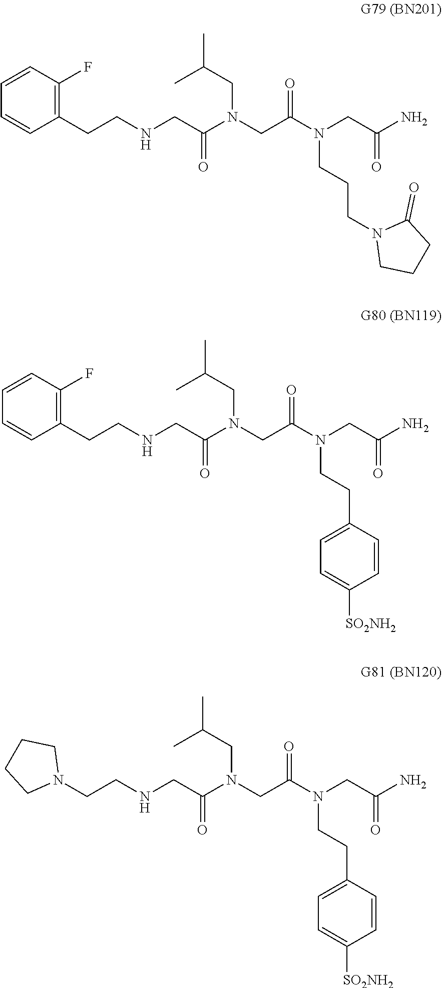 [SMILES: CC(C)CN(CC(=O)N(CCCN1CCCC1=O)CC(N)=O)C(=O)CNCCc1ccccc1F ; CC(C)CN(CC(=O)N(CCc1ccc(S(N)(=O)=O)cc1)CC(N)=O)C(=O)CNCCc1ccccc1F ; CC(C)CN(CC(=O)N(CCc1ccc(S(N)(=O)=O)cc1)CC(N)=O)C(=O)CNCCN1CCCC1]

Clause 5. The combination according to any of clauses 1-4, wherein the compound of formula (IV), or a pharmaceutically or veterinary acceptable salt thereof is selected from the group consisting of dimethyl fumarate, monoethyl fumarate, sodium fumarate and iron (II) fumarate.

Clause 6. The combination according to any of clauses 1-5, wherein the S1PR modulator is selected from the group consisting of fingolimod, siponimod, ozazimod, ponesimod, and ceralifimod.

Clause 7. The combination according to any of the clauses 1-6, wherein the STAT3 inhibitor is selected from the group consisting of 2-Hydroxy-4-[[[[(4-methylphenyl)sulfonyl]oxy]acetyl]amino]-benzoic acid, (S,E)-3-(6-Bromopyridin-2-yl)-2-cyano-N-(1-phenylethyl)acrylamide, 4-((3-(Carboxymethylsulfanyl)-4-hydroxy-1-naphthyl)sulfamoyl)benzoic acid, STAT3 Inhibitor Peptide of SEQ ID NO: 1, 6-Nitrobenzo[b]thiophene-1,1-dioxide, Ethyl-1-(4-cyano-2,3,5,6-tetrafluorophenyl)-6,7,8-trifluoro-4-oxo-1,4-dihydroquinoline-3-carboxylate, 5,15-Diphenylporphyrin, PIAS3 protein, N-(5-(Furan-2-yl)-1,3,4-oxadiazol-2-yl)-2-phenylquinoline-4-carboxamide, STAT3 Inhibitor XII SPI of SEQ ID NO: 2, and N-(1′,2-Dihydroxy-1,2′-binaphthalen-4′-yl)-4-methoxybenzenesulfonamide.

Clause 8. The combination according to any of clauses 1-7, which is selected from:
the compound of formula (I) [N-(2-(2′-Fluorophenyl)ethyl)glycyl]-[N-(2-methylpropyl)glycyl]-N-[3-(2′-oxopyrrolidinyl)-propyl]glycinamide, and dimethyl fumarate; or alternatively,
the compound of formula (I) [N-(2-(2′-Fluorophenyl)ethyl)glycyl]-[N-(2-methylpropyl)glycyl]-N-[3-(2′-oxopyrrolidinyl)-propyl]glycinamide, and fingolimod; or alternatively,
the compound of formula (I) [N-(2-(2′-Fluorophenyl)ethyl)glycyl]-[N-(2-methylpropyl)glycyl]-N-[3-(2′-oxopyrrolidinyl)-propyl]glycinamide, and 2-Hydroxy-4-[[[[(4-methylphenyl)sulfonyl]oxy]acetyl]amino]-benzoic acid.

Clause 9. The combination according to any of clauses 1-8 comprising:
a) a compound of formula (I), or a pharmaceutically or veterinary acceptable salt thereof, and
b) one or more drugs selected from the group consisting of: i) a compound of formula (IV), or a pharmaceutically or veterinary acceptable salt thereof, ii) a S1PR modulator, and iii) a STAT3 inhibitor, as previously defined,
   wherein the amount of a) and the amount of b) in combination are therapeutically effective.

Clause 10. The combination according to any of clauses 1-9, wherein the amount of a) separately is a subtherapeutic amount; and the amounts of a) and b) in combination are therapeutically effective.

Clause 11. The combination according to any of clauses 1-9, wherein the amount of b) separately is a subtherapeutic amount; and the amounts of a) and b) in combination are therapeutically effective.

Clause 12. The combination according to any of clauses 1-9, wherein the amount of a) and the amount of b) separately are subtherapeutic amounts; and the amounts of a) and b) in combination are therapeutically effective.

Clause 13. A single pharmaceutical or veterinary composition which comprises a therapeutically effective amount of:
a) a compound of formula (I) or a pharmaceutically or veterinary acceptable salt thereof

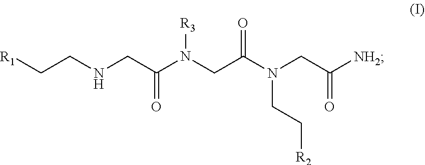

and
b) one or more drugs selected from the group consisting of a compound of formula (IV) or a pharmaceutically or veterinary acceptable salt thereof, a S1PR modulator, and a STAT3 inhibitor

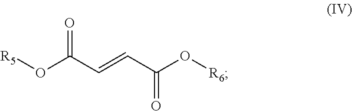

together with one or more pharmaceutically or veterinary acceptable excipients or carriers; wherein the compound of formula (I) and one or more drugs are as defined in any of the clauses 1-9, and wherein the amount of a) and the amount of b) in combination are therapeutically effective.

Clause 14. A package or kit of parts comprising:
i) a first pharmaceutical or veterinary composition which comprises an amount of a compound of formula (I) as defined in any of clauses 1-9, or a pharmaceutically or veterinary acceptable salt thereof, together with one or more pharmaceutically or veterinary acceptable excipients or carriers; and
ii) a second pharmaceutical or veterinary composition which comprises an amount of one or more drugs selected from the group consisting of a compound of formula (IV), or a pharmaceutically or veterinary acceptable salt thereof, a S1PR modulator, and a STAT3 inhibitor, together with one or more pharmaceutically or veterinary acceptable excipients or carriers;
wherein the first and second compositions are separate compositions, and wherein the amount of the compound of formula (I) of i) and the amount of one or more drugs of ii) in combination are therapeutically effective.

Clause 15. A combination as defined in any of clauses 1-12, a single pharmaceutical or veterinary composition as defined in clause 13, or a package or kit of parts as defined in clause 14, for use in the treatment and/or prevention of an inflammatory neurological disease or condition which can result in the destruction or degeneration of axons or myelin in a subject in need thereof.

### CITATION LIST

### Patent Literature

EP 2611775

### Non Patent Literature

Patrick Vermersch et al., “Sphingosine-1-phosphate Receptor Modulators in Multiple Sclerosis”; European Neurological Review-2018; 13(1):25-30
Guidance for Industry estimating the maximum safe starting dose in initial clinical trials for therapeutics in adult healthy volunteers; FDA, CDER, July 2005
Kezuka et al, Analysis of the pathogenesis of experimental autoimmune optic neuritis. J Biomed Biotechnol. 2011; 2011:294046.
Guo et al, 2009; Guo J, Li H, Yu C, Liu F, Meng Y, Gong W, Yang H, Shen X, Ju G, Li Z, Wang J. Decreased neural stem/progenitor cell proliferation in mice with chronic/nonremitting experimental autoimmune encephalomyelitis. Neurosignals. 2010; 18(1):1-8.

I claim:

1. A method of treating optic neuritis comprising administering to a human in need thereof:
(a) a first pharmaceutical composition comprising: 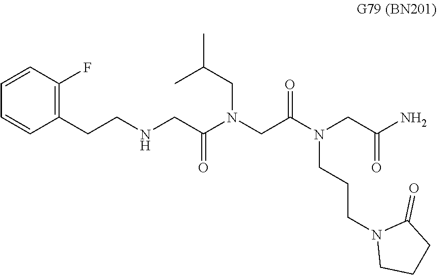
or a pharmaceutically acceptable salt thereof comprising at least one pharmaceutically acceptable excipient;
in a therapeutically effective combination with
(b) a second pharmaceutical composition of dimethyl fumarate or monomethyl fumarate, or a mixture thereof, or a pharmaceutically acceptable salt thereof, comprising at least one pharmaceutical excipient.

2. The method of claim 1, wherein the pharmaceutical composition of BN201 or a pharmaceutically acceptable salt thereof is administered parenterally.

3. The method of claim 2, wherein the pharmaceutical composition of BN201 or a pharmaceutically acceptable salt thereof is administered intravenously.

4. The method of claim 1, wherein the pharmaceutical composition of dimethyl fumarate, monomethyl fumarate or a mixture thereof is administered orally.

5. The method of claim 4, wherein the pharmaceutical composition is a tablet.

6. The method of claim 1, wherein the compound of (b), or a pharmaceutically acceptable salt thereof, is monomethyl fumarate.

7. The method of claim 1, wherein the compound of (b), or a pharmaceutically acceptable salt thereof, is dimethyl fumarate.

8. The method of claim 1, wherein the pharmaceutical combination treats degeneration of axons.

9. The method of claim 1, wherein the pharmaceutical combination treats degeneration of myelin.

10. The method of claim 1, that treats the associated inflammatory neurological condition.

11. The method of claim 1, wherein the pharmaceutical composition of (a) is administered once a day.

12. The method of claim 1, wherein the pharmaceutical composition of (a) is administered twice a day.

13. The method of claim 1, wherein the pharmaceutical composition of (b) is administered once a day.

14. The method of claim 1, wherein the pharmaceutical composition of (b) is administered twice a day.

15. The method of claim 11, wherein the pharmaceutical composition of (b) is administered once a day.

16. The method of claim 11, wherein the pharmaceutical composition of (b) is administered twice a day.

17. The method of claim 1, wherein dimethyl fumarate is administered at about 120 mg twice a day.

18. The method of claim 1, wherein dimethyl fumarate is administered at about 240 mg twice a day.

19. The method of claim 1, wherein BN201 is administered at 0.5 mg/kg to 25 mg/kg.
